SEQUENCE LISTING (40 sequences)

SEQ ID NO: 1 - protein, 9 aa, Homo sapiens
NVWIGLHDP

SEQ ID NO: 2 - protein, 166 aa, Homo sapiens
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 3 - protein, 166 aa, Homo sapiens
MAQTNSFFMLISSLMFLSLSQGQESQTELPNPRISCPEGTNAYRSYCYYFNEDPETWVDADLYCQNMNSGNLVSVLTQAEGAFVASLIKESSTDDSNVWIGLHDPKKNRRWHWSSGSLVSYKSWDTGSPSSANAGYCASLTSCSGFKKWKDESCEKKFSFVCKFKN

SEQ ID NO: 4 - protein, 8 aa, Homo sapiens
VWIGLHDP

SEQ ID NO: 5 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 6 - protein, 919 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLFKTRLPHDKTKCFKFI

SEQ ID NO: 7 - protein, 7 aa, Homo sapiens
WIGLHDP

SEQ ID NO: 8 - protein, 8 aa, Homo sapiens
WIGLHDPT

SEQ ID NO: 9 - protein, 20 aa, Homo sapiens
CKKSIENAKQDLLQLKNVIS

SEQ ID NO: 10 - protein, 158 aa, Homo sapiens
MASRSMRLLLLLSCLAKTGVLGDIIMRPSCAPGWFYHKSNCYGYFRKLRNWSDAELECQSYGNGAHLASILSLKEASTIAEYISGYQRSQPIWIGLHDPQKRQQWQWIDGAMYLYRSWSGKSMGGNKHCAEMSSNNNFLTWSSNECNKRQHFLCKYRP

SEQ ID NO: 11 - protein, 14 aa, Homo sapiens
IGLHDPTQGTEPNG

SEQ ID NO: 12 - protein, 15 aa, Mesocricetus auratus
IGLHDPSHGTLPNGS

SEQ ID NO: 13 - protein, 333 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPP

SEQ ID NO: 14 - protein, 9 aa, Homo sapiens
VWIGLHDPT

SEQ ID NO: 15 - protein, 15 aa, Artificial Sequence
  synthetic
  MOD_RES (1)..(1): ACETYLATION
  MOD_RES (15)..(15): AMIDATION
IGLHDPSHGTLPNGS

SEQ ID NO: 16 - protein, 16 aa, Artificial Sequence
  synthetic
CIGLHDPSHGTLPNGS

SEQ ID NO: 17 - protein, 16 aa, Artificial Sequence
  synthetic
  DISULFID (1)..(1): Homodimer formed via the creation of a disulfide bond between the cysteine residues of the individual monomers of SEQ ID NO17.
CIGLHDPSHGTLPNGS

SEQ ID NO: 18 - protein, 16 aa, Artificial Sequence
  synthetic
  MOD_RES (1)..(1): ACETYLATION
  DISULFID (1)..(1): Homodimer formed via the creation of a disulfide bond between the cysteine residues of the individual monomers of SED ID NO 18.
  MOD_RES (16)..(16): AMIDATION
CIGLHDPSHGTLPNGS

SEQ ID NO: 19 - protein, 16 aa, Artificial Sequence
  synthetic
  MOD_RES (1)..(1): N-terminal cysteine covalently bonded to a dimeric maleimide activated 40Kd PEG construct.
CIGLHDPSHGTLPNGS

SEQ ID NO: 20 - protein, 16 aa, Artificial Sequence
  synthetic
  MOD_RES (1)..(1): ACETYLATION
  MOD_RES (1)..(1): N-terminal cysteine covalently bonded to a dimeric maleimide activated 40Kd PEG construct.
  MOD_RES (16)..(16): AMIDATION
CIGLHDPSHGTLPNGS

SEQ ID NO: 21 - protein, 15 aa, Artificial
  synthetic
  ACETYLATION (1)..(1)
  PEGYLATION (15)..(15): conjugation with 40-kDa poly (ethylene glycol) (PEG)-maleimide
  AMIDATION (15)..(15)
IGLHDPSHGTLPNGS

SEQ ID NO: 22 - protein, 15 aa, Artificial
  synthetic
  PEGYLATION (1)..(1): conjugation with 40-kDa poly (ethylene glycol) (PEG)-maleimide-3-mercaptopropionic acid
  AMIDATION (15)..(15)
IGLHDPSHGTLPNGS

SEQ ID NO: 23 - protein, 17 aa, Artificial
  synthetic
  MISC_FEATURE: Cyclic amide bond between side chain of Asp on position 1 and Lys on position 17
  ACETYLATION (1)..(1)
  AMIDATION (17)..(17)
DIGLHDPSHGTLPNGSK

SEQ ID NO: 24 - protein, 14 aa, Artificial
  synthetic
  ACETYLATION (1)..(1)
  AMIDATION (14)..(14)
IGLHDPTQGTEPNG

SEQ ID NO: 25 - protein, 14 aa, Artificial
  synthetic
  ACETYLATION (1)..(1)
  PEGYLATION (14)..(14): conjugation with 40-kDa poly (ethylene glycol) (PEG)-maleimide
  AMIDATION (14)..(14)
IGLHDPTQGTEPNG

SEQ ID NO: 26 - protein, 14 aa, Artificial
  synthetic
  PEGYLATION (1)..(1): conjugation with 40-kDa poly (ethylene glycol) (PEG)-maleimide-3-mercaptopropionic acid
  AMIDATION (14)..(14)
IGLHDPTQGTEPNG

SEQ ID NO: 27 - protein, 16 aa, Artificial
  synthetic
  MISC_FEATURE: Cyclic amide bond between side chain of Asp on position 1 and Lys on position 16
  ACETYLATION (1)..(1)
  AMIDATION (16)..(16)
DIGLHDPTQGTEPNGK

SEQ ID NO: 28 - protein, 7 aa, Artificial
  synthetic
  ACETLYATION (1)..(1)
  AMIDATION (7)..(7)
WIGLHDP

SEQ ID NO: 29 - protein, 7 aa, Artificial
  synthetic
  ACETYLATION (1)..(1)
  PEGYLATION (7)..(7): conjugation with 40-kDa poly (ethylene glycol) (PEG)-maleimide
  AMIDATION (7)..(7)
WIGLHDP

SEQ ID NO: 30 - protein, 7 aa, Artificial
  synthetic
  PEGYLATION (1)..(1): conjugation with 40-kDa poly (ethylene glycol) (PEG)-maleimide-3-mercaptopropionic acid
  AMIDATION (7)..(7)
WIGLHDP

SEQ ID NO: 31 - protein, 9 aa, Artificial
  synthetic
  MISC_FEATURE: Cyclic amide bond between side chain of Asp on position 1 and Lys on position 9
  ACETYLATION (1)..(1)
  AMIDATION (9)..(9)
DWIGLHDPK

SEQ ID NO: 32 - protein, 8 aa, Artificial
  synthetic
  ACETLYATION (1)..(1)
  AMIDATION (8)..(8)
VWIGLHDP

SEQ ID NO: 33 - protein, 8 aa, Artificial
  synthetic
  ACETYLATION (1)..(1)
  PEGYLATION (8)..(8): conjugation with 40-kDa poly (ethylene glycol) (PEG)-maleimide
  AMIDATION (8)..(8)
VWIGLHDP

SEQ ID NO: 34 - protein, 8 aa, Artificial
  synthetic
  PEGYLATION (1)..(1): conjugation with 40-kDa poly (ethylene glycol) (PEG)-maleimide-3-mercaptopropionic acid
  AMIDATION (8)..(8)
VWIGLHDP

SEQ ID NO: 35 - protein, 10 aa, Artificial
  synthetic
  MISC_FEATURE: Cyclic amide bond between side chain of Asp on position 1 and Lys on position 10
  ACETYLATION (1)..(1)
  AMIDATION (10)..(10)
DVWIGLHDPK

SEQ ID NO: 36 - protein, 9 aa, Artificial
  synthetic
  ACETYLATION (1)..(1)
  AMIDATION (9)..(9)
NVWIGLHDP

SEQ ID NO: 37 - protein, 9 aa, Artificial
  synthetic
  ACETYLATION (1)..(1)
  PEGYLATION (9)..(9): conjugation with 40-kDa poly (ethylene glycol) (PEG)-maleimide
  AMIDATION (9)..(9)
NVWIGLHDP

SEQ ID NO: 38 - protein, 9 aa, Artificial
  synthetic
  PEGYLATION (1)..(1): conjugation with 40-kDa poly (ethylene glycol) (PEG)-maleimide-3-mercaptopropionic acid
  AMIDATION (9)..(9)
NVWIGLHDP

SEQ ID NO: 39 - protein, 11 aa, Artificial
  synthetic
  MISC_FEATURE: Cyclic amide bond between side chain of Asp on position 1 and Lys on position 11
  ACETYLATION (1)..(1)
  AMIDATION (11)..(11)
DNVWIGLHDPK

SEQ ID NO: 40 - protein, 165 aa, Artificial
  synthetic
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

(19) United States
(12) Patent Application Publication
(10) Pub. No.: US 2020/0054716 A1
(43) Pub. Date: Feb. 20, 2020

(54) COMPOSITIONS AND METHODS FOR TREATING OR PREVENTING TYPE 1 DIABETES USING A BIOLOGIC RESPONSE MODIFIER IN COMBINATION WITH ONE OR MORE ISLET OR BETA CELL REGENERATION OR REPLACEMENT THERAPIES

(71) Applicant: Claresa Levetan, Philadelphia, PA (US)

(72) Inventor: Claresa Levetan, Philadelphia, PA (US)

(21) Appl. No.: 16/675,500

(22) Filed: Nov. 6, 2019

Related U.S. Application Data
(62) Division of application No. 15/815,318, filed on Nov. 16, 2017.

(51) Int. Cl. A61K 38/21; A61K 38/10; A61K 38/17

(52) U.S. Cl. CPC A61K 38/212; A61K 9/0053; A61K 38/1709; A61K 38/10

(57) ABSTRACT

Methods and pharmaceutical compositions of a unique Biologic Response Modifier (BRM) that does not suppress the immune system, yet provides protection of beta cells in those with type 1 diabetes and those at risk for type 1 diabetes are described. The methods include utilization of BRMs in combination with islet neogenesis therapies, beta regeneration therapies, islet, beta cell or stem cell transplants, or devices housing islets, beta cells or stem cells for treatment and prevention of type 1 patients and related conditions. The compositions and methods provide for beta cell protection from autoimmune attack for prevention or delay in the onset of type 1 diabetes. The BRM may be used in conjunction with immunosuppressive agents. The BRM may also be used in other conditions found among patients with type 1 diabetes and their relatives for whom there is no treatment or current therapy is unsuccessful.

### CROSS-REFERENCE TO RELATED APPLICATION

The present application is a Divisional Application of U.S. Non-Provisional Utility patent application Ser. No. 15/815,318 which was filed on Nov. 16, 2017.

### REFERENCE TO SEQUENCE LISTING SUBMITTED IN COMPUTER READABLE FORM

The present application contains a Sequence Listing, which has been submitted in ASCII format by way of EFS-Web and is hereby incorporated by reference herein in its entirety. The ASCII file was created Oct. 6, 2017 and named C.LEV-018_SEQ, which is 29,552 bytes in size and which is identical to the paper copy filed with this application.

### FIELD OF THE INVENTION

Embodiments of the present invention relate to novel therapies, pharmaceutical compositions and methods for treating type 1 diabetes and related conditions, including new onset type 1 diabetes, pre-existing type 1 diabetes, and pre-Type 1 Diabetes. In particular, embodiments of the invention relate to treating conditions and populations in which there are positive autoimmune antibodies associated with the later onset of type 1 diabetes, including first or second degree relatives of with type 1 diabetes and latent autoimmune diabetes of adulthood (LADA). In embodiments, the positive autoimmune antibodies, may include, but are not limited to Zinc Transporter 8 (ZnT8) Antibodies, islet cell antibody/Protein tyrosine phosphatase islet antigen-2 (IA2), Glutamic Acid Decarboxylase (GAD65) Antibodies, antibodies to insulinoma-associated antigen-2 (ICA512), insulin (micro-IAA [mIAA]), and insulin antibodies (IAA). Embodiments of the invention may also be used to treat patients without autoimmune antibodies associated with diabetes, those with altered glucose homeostasis associated with autoimmune attack on beta cells, relatives of type 1 diabetes patients with or without the presence of autoimmune antibodies or glucose alterations associated with the development of diabetes. In embodiments, the novel therapies, pharmaceutical compositions and methods utilize a Biological Response Modifier (BRM), which modifies the body's attack on insulin-producing beta cells without suppressing the immune system or producing adverse effects. Further, embodiments may use a BRM alone or in combination with other beta cell regeneration and/or replacement therapies, including islet neogenesis agents, beta regeneration agents, islet or beta cell transplants, stem cell transplants, or implantations of a devices containing islets, beta cells or stem cells. Further, embodiments may use a BRM alone or in combination with one or more immune tolerance agents to prevent type 1 diabetes from developing, prevent autoimmune attack on beta cells, as well as achieve insulin independence among patients with type 1 diabetes.

Additionally, the present inventor presents data suggesting that a BRM such as oral interferon alfa-2a may be used in the treatment of other autoimmune diseases, some of which are associated with type 1 diabetes including, but not limited to, multiple sclerosis, amyotrophic lateral sclerosis (ALS), frontotemporal dementia, and other conditions, which may have an autoimmune basis. Neurological or rheumatologic conditions or diagnoses for which there is a suspected autoimmune component, such as Hashimoto's Hypothyroidism and Multiple Sclerosis, may be treated with oral interferon alfa-2a to help limit or delay progressive symptoms, particularly when other treatment regimens are exhausted. The use of oral interferon alfa-2a may be used alone or in combination with other therapies to prevent the progression of symptoms in these conditions, which to date lack adequate therapies.

Although trials were conducted in non-obese diabetic (NOD) mice and in three human studies among new onset type 1 diabetes, as of 2017, both of the lead investigators (Brod and Rother) in personal correspondence with the present inventor have not pursued further study of oral interferon alfa-2a for diabetes in more than a decade because it did not render any patients insulin independent.

### BACKGROUND OF THE INVENTION

Embodiments of the invention address the critical unmet need of type 1 diabetes in which there have been no significant changes in treatment since 1922 when insulin was first used to save the life of a young boy. The present inventor disagrees with current convention accepted, as dogma over the past forty years that type 1 diabetes, in man, is a disease solely of autoimmunity.

As a unique contribution to the art, the present inventor has microscopically evaluated the distinct differences of the Islets of Langerhans in humans and in mice and has shown that the marked differences between Islets of mice and man make it clear why more than 300 studies have reversed diabetes in the “type 1 diabetes mouse model,” known as the NOD mouse, while none of the successful treatments in NOD mice have led to insulin independence among type 1 human patients with the disease (see Levetan et al., Endocr Pract. 2013; 19(2):301-12). The present inventor has also described the genomic and transcription factor differences between islet neogenesis in man and the process of beta cell regeneration (see Levetan C. J Diabetes. 2010; 2(2):76-84).

The current understanding in the art that Islets of Langerhans are synonymous with beta cells is perpetuated in the scientific literature including recent articles published in leading peer-reviewed journals, which still refer to the Islets of Langerhans as beta/islets and islet beta cells. The present inventor has clearly demonstrated that, in contrast to mice, the beta cell in humans is an intricate and integral part of a complex network of interwoven cells within the Islets of Langerhans. This is shown in FIG. 1A. The red cells are beta cells staining for insulin, while green cells are alfa-2a cells, staining for glucagon and blue cells are delta cells staining for somatostatin. The black holes in the human islet are blood vessels lined with smooth muscle and innervated by the nervous system.

The present inventor is the first to posit that the distinctions between islets of mice and man explain why islets of mice, whose central islet core is made up completely of beta cells, have a faster turnover rate than in man because of the mouse's shorter life span of 12-18 months and the mouse's continuous eating patterns. The present inventor is also the only one who has hypothesized the reason why so many types of immune therapies work in NOD mouse models, but have failed to reverse diabetes in new onset type 1 diabetes human patients.

Despite breeding of NOD mice as an animal model for type 1 diabetes that has a leukocytic infiltrate of the pancreatic islets, this model does not simulate the autoimmune attack on beta cells that occur in man. The present inventor is the first to disagree with the current definition of type 1 diabetes as only an autoimmune disease, which helps better understand and clarify why single or combination immune therapies, which reverse diabetes in NOD mice, have not worked in man.

In humans, beta cells are intricately woven inside the islet, and require a process of islet neogenesis for new beta cells rather than beta cells regeneration from existing beta cells. The present inventor is the first to demonstrate that the transcription factors required for islet neogenesis are different than those required for beta cell regeneration from existing beta cells (see Levetan, J Diabetes. 2010; 2(2):76-84).

The present inventor uniquely hypothesizes that in man, diabetes is a disease of autoimmunity as well as a lack of beta cell regeneration, even in an immune-muted milieu. This is in contrast to the currently held view by the Diabetes Immune Tolerance Network, in collaboration with the Juvenile Diabetes Research Foundation, and Type 1 Diabetes Combination Therapy Assessment Group who have only considered that type 1 diabetes is a disease of autoimmunity and could and should only be treated by stated autoimmune therapies proposed by their consensus panel (see Matthews et. Al., Clin Exp Immunol. 2010; 160(2):176-84). The consensus panel of type 1 diabetes experts states and continues to state that type 1 diabetes is only an autoimmune disease and only therapies, which are immune suppressants will cure this disease.

The present inventor has identified the differences between islets and beta cell distribution and vascularization between mice and men, including the finding that human islets house four types of cells that secrete five hormones (insulin, glucagon, somatostatin, pancreatic polypeptide and islet ghrelin), all of which are necessary for normal glucose homeostasis in man. In contrast to the mouse, each of these cell types is interwoven within the human islet. Based on these findings, unique to the art, the present inventor identifies type 1 diabetes as a disease of autoimmunity.

The present inventor has identified therapies for islet neogenesis (see U.S. Pat. Nos. 7,393,919; 7,714,103; 7,989,415; 8,211,430; 8,785,400; 8,808,689; 9,321,812; and 9,511,110), in contrast to potential therapies that regenerate beta cells from existing beta cells or those that generate beta cells ex-vivo and transfer them to a patient with type 1 diabetes. Embodiments of the present invention specify that an oral BRM may be used with any therapy or combination of therapies in the field of type 1 diabetes to prevent autoimmune attack of insulin producing cells. To date, there is no such oral therapy, nor has there been continued research in the field for more than a decade for the usage of an agent such as oral interferon alfa-2a to protect type 1 diabetes patient's insulin producing cells, either alone or in combination with other beta cell regeneration and/or replacement therapies such as islet regeneration/neogenesis agents, beta cell regeneration therapies, islet transplants, beta cells transplants from cadaveric or ex-vivo generated beta cells, stem cell transplants for type 1 diabetes, or devices encapsulating islets, beta cells or stem cells.

Despite many advances in the field of diabetes, both men and women with type 1 diabetes still face shortened lifespans of more than a decade (11 years shorter for men and 13 years shorter for women) (see Livingstone JAMA. 2015:6; 313(1):37-44). The largest percentage of loss of life in those under the age of 50 with type 1 diabetes is directly due to either critically low or critically high glucose levels. In the United States, 208,000 people younger than age 20 have type 1 diabetes, with more than 15,000 new cases of type 1 diabetes diagnosed each year among adults and 16,000 new cases among children.

Children with type 1 diabetes are five times more likely to be hospitalized than children without the disease. Further, 73.5% of child hospitalizations in the US are for uncontrolled diabetes, with more than 32,000 children under the age of 17 hospitalized each year for life-threatening diabetic ketoacidosis (see https://www.cdc.gov/diabetes/statistics/hosp/kidtable1.htm and Sayers, A., et al., “Evidence for a persistent, major excess in all cause admissions to hospital in children with type-1 diabetes: results from a large Welsh national matched community cohort study”, BMJ Open 2015; 5:e005644. doi:10.1136/bmjopen-2014-005644).

The number of adults hospitalized annually in the U.S. has nearly doubled over a decade with more than 168,000 adult patients hospitalized with type 1 diabetes hospitalized with life-threatening type 1 diabetes per year in the US among type 1 patients with 300,000 adults visiting U.S. emergency rooms annually for hypoglycemia with 145,000 requiring hospitalization. (https://www.cdc.gov/diabetes/pdfs/data/statistics/national-diabetes-statistics-report.pdf) There has been a 3-5% rise each year in the number of new cases of type 1 diabetes, which has now become one of the most common chronic diseases of childhood (see Gale, Diabetes 2002; 51(12): 3353-3361).

Unique to the field of diabetes treatment is the combination of beta cell regeneration and/or replacement therapies such as an islet regeneration or beta regeneration agents, islet, beta or stem cell transplant or devices that houses beta cells, islets or stems cells with a unique, safe oral BRM, which defined in this specification as a therapy that modifies the immune systems attack on insulin-producing beta cells, but does not suppress the body's immune system and is not associated with adverse effects beyond that of a placebo.

Such a combination therapy is completely new to the art, and was never previously mentioned as a therapy for type 1 diabetes or as a preventive therapy for those at risk for the development of type 1 diabetes. Only therapies that are immune suppressive agents have been considered alone or in combination with one another as potential diabetes cures, and only the present inventor proposes a BRM for Type 1 diabetes, Pre-Type 1 diabetes, and those with no glucose abnormalities but with antibodies that are highly predictive of the development of diabetes.

To date, hundreds of therapies have been used to try to prevent the onset of type 1 diabetes in those at risk for the disease, especially in first degree relatives who exhibit one or more antibodies such as Zinc Transporter 8 (ZnT8) Antibodies, islet cell antibody/Protein tyrosine phosphatase islet antigen-2 (IA2), Glutamic Acid Decarboxylase (GAD65) Antibodies, antibodies to insulinoma-associated antigen-2 (ICA512), insulin (micro-IAA [mIAA]), and insulin antibodies (IAA). In such patients, numerous agents have been studied in clinical trials to delay or prevent the onset of type 1 diabetes. These treatments have included therapies such as oral insulin, low dosages of insulin prior to the onset of disease, and vitamin D therapy.

The present inventor has patented a number of peptides from the Reg gene family that have been shown to arise during acute pancreatic injury and that transform pancreatic ductal progenitor cells within the adult pancreas into new islets (see Kapur et al., Islets 2012; 4(1):40-8). Many groups from around the world have confirmed that the Reg peptides patented by the present inventor not only transform human pancreatic ductal tissue into new islets in vivo, but have also demonstrated that the newest progenitor cells that form into new small islet in vitro stain for these Reg peptides (see Guo 2010, Scientific Sessions of the American Diabetes Association).

The present inventor has uniquely examined why more than 300 studies in NOD mice have succeeded in curing type 1 diabetes, while none have in humans. Despite dozens of potential therapies, which have primarily consisted of intravenous and subcutaneous injections of immune agents for reversal or type 1 diabetes in man, none have included an oral BRM in conjunction with a therapy that generates islets or beta cells, or devices that encapsulate islets, beta cells or mesenchymal or embryonic stem cells.

The present inventor presents a new therapy in the field that specifically provides usage of a safe and effective oral BRM for type 1 diabetes. Based on this critical unmet need, the present specification identifies a unique combination of a safe and effective BRM, alfa-2a interferon, with an islet regeneration agent or beta regeneration agent for the purpose of insulin independence among type 1 patients. The present inventor presents the combination of a safe islet neogenesis therapy combined with safe and effective immune protection with the potential achievement of insulin independence among type 1 patients.

Over the past four decades, when the etiology of type 1 diabetes was found to be autoimmune in nature, new discoveries have conceptually changed the way type 1 diabetes in humans is viewed, raising new questions about how this disease will be reversed in humans. The present inventor answers these questions in this specification:

Why usage of immune agents alone or usage of regenerative therapies alone reverse diabetes in type 1 diabetic mouse models (NOD), but not in man?

Why don't beta cells develop among new onset type 1 patients when single or multiple immune agents are given at the onset of the disease?

How to harness the discoveries by research teams around the world that progenitor cells exist in the pancreas that can be triggered to form new insulin-producing cells without transplants?

What is the optimal way to protect beta cells, especially newly formed beta cells, from the autoimmune attack?

The present inventor has discovered why many successful trials in NOD mice (type 1 diabetes mouse models) have not translated into type 1 diabetes therapies in man. The lack of success in translating the ability of numerous therapeutic agents from a variety of different classes to reverse type 1 diabetes in NOD mice, but not in man, has been discovered by the present inventor as a result of understanding the unique composition and greater complexity of human islets compared to rodent islets (see Levetan et al., Endocr Pract. 2013; 19(2):301-12).

In humans, more than 70% of beta cells are directly contiguous with alfa-2a, gamma, delta and epsilon cells within the islets as shown in FIG. 1A. In rodents the beta cells form most of the islet with a small mantle of alfa-2a and delta cells around the beta cells as depicted in FIG. 1B. Therefore, in man, regeneration of beta cells is a much more complex process than it is in mice which have a centralized core of beta cells with a more rapid turnover time than those in man.

These differences are also explained by the present inventor as a result of the vast differences in the human lifespan compared to the one-year lifespan of rodents as well as their continuous eating patterns; in contrast, the islets of man evolved to enable man to survive both in times of feast and famine, and this may explain why beta cell turnover time is faster in rodents than in man. By understanding the unique differences between the islets of mice and men, the present inventor has developed a new and unique understanding that type I diabetes in not only a disease of autoimmunity, but also due to a lack of regeneration of beta cells, even in an immune muted milieu.

The present inventor uniquely posits the use of islet and beta cell therapies such as an islet neogenesis agent, beta regeneration agent, islet transplant, beta cell transplant or stem cell transplant with the usage of an oral BRM, such as alfa-2a interferon. In embodiments, the BRM is administered at a dosage of 1/10,000 the typical subcutaneous dosage for diseases such as hairy cell leukemia or hepatitis C, such as at a hormetic dosage of about 5000 IU orally to protect beta cell mass. The combination of such therapies may result in insulin independence among new onset type 1 patients, unlike previous combinations of immune suppressant agents.

The present inventor has shown the efficacy of Reg gene peptide therapies in regenerating insulin-producing cells in vivo among both type 1 and type 2 diabetes patients. To date, more than 50 peer-reviewed articles have confirmed that Reg proteins and peptides transform progenitor cells within the pancreas into functioning islets, including human pancreatic tissue, without the need for transplantation. Additionally, Reg peptides upregulate transcription factors associated with islet neogenesis in vivo including Pdx-1, Ngn3, NeuroD, IA-1, MafA, Nkx6.1, Sox9 and Ins (see Kapur et al., Islets. 2012; 4(1):40-8; Assouline-Thomas et al., Differentiation. 2015; 90(4-5):77-90; Levetan C. J Diabetes. 2010; 2(2):76-84).

The present inventor has patented (see U.S. Pat. No. 9,321,812) an optimized peptide which has been used in Phase 2B trials in type 1 and 2 diabetes which have demonstrated significant reduction in hemoglobin A1C among type 2 patients and a significant rise in stimulated C-peptide among type 1 patients having the disease for 20 years. This optimized peptide has a six-fold higher stability in human plasma than the native peptide, with a resulting increase in insulin and transcription factors associated with islet neogenesis (see Kapur R, Højfeldt T W, Højfeldt T W, et al, Short-term effects of INGAP and Reg family peptides on the appearance of small β-cells clusters in non-diabetic mice. Islets. 2012 January-February; 4(1):40)). Additionally, the present inventor has patented a number of islet neogenesis peptides or optimized peptides (see U.S. Pat. Nos. 7,714,103; 7,393,919; 7,989,415; 8,383,578; 8,816,047; 8,785,400; 9,133,440; 9,511,110; 8,911,776; 8,829,158; 8,816,047; and 9,321,812).

Specifically novel to the field is the combination of an islet neogenesis agent for in vivo usage with an oral BRM, which is not acting as an immune suppressant. Additionally novel are dosages and routes of administration of the BRM, which do not render a patient with type 1 diabetes at risk for immunosuppression. The present inventor specifically proposes that type 1 diabetes is a disease of autoimmunity characterized as a potential interferon immunodeficiency syndrome and that in man, oral interferon acts not as an immune suppressant, but as a BRM to protect beta cell mass from immune destruction. This is in contrast to dozens of immunosuppressants that reduce attack on beta cells with side effects including immunosuppression resulting in increased risk for infection or cancers. A BRM embodiment of the present invention, interferon alfa-2a, has not produced side effects in three human trials among new onset type 1 diabetes patients aged 2-25 years old and has resulted in significant preservation of beta cell mass compared to controls after 12 months of treatment.

The present inventor uniquely combines an effective and safe BRM with therapies designed to increase beta cell mass, such as a beta regeneration agent, islet neogenesis agent, islet or beta cell transplantation. The present inventor has found a unique BRM for type 1 diabetes, which had already been eliminated as possibly therapy for type 1 diabetes by 2010 and is not included as a potential therapy or in a combination of therapies by the Diabetes Immune Tolerance Network, in collaboration with the Juvenile Diabetes Research Foundation, and Type 1 Diabetes Combination Therapy Assessment Group (see Matthews et. Al., Clin Exp Immunol. 2010; 160(2): 176-84).

Research in the field decades ago used oral interferon at 1/10,000 of the typical dosage that is used subcutaneously for treatment of conditions such as Hairy Cell Leukemia, Multiple Sclerosis and Hepatitis C. Researchers conducting studies in both NOD mice and in three human trials among new onset type 1 diabetes patients did not consider oral interferon efficacious enough to pursue further study in the field of diabetes.

Unique to the art, the present inventor identifies the potential to use oral interferon as a BRM, due to its safety and potential to protect beta cell mass as measured by stimulated C-peptide. It is not understood in the current art that there is a potential to use oral interferon alfa-2a with an agent, such as ones discovered by the present inventor to regenerate new islets from progenitors within the adult pancreas. The present inventor uniquely identifies the potential for the usage of oral interferon alfa-2a for insulin independence among type 1 patients when used with one or more therapies such as an islet neogenesis agent, beta regeneration agent, islet, beta cell or stem cell transplant, or encapsulation of islets, beta cells or stem cells in a patient with type 1 diabetes. New to the art is the use of oral interferon alfa-2a to prevent or delay the onset of type 1 diabetes, including on those who are at risk for developing type 1 diabetes (who may be identified by the number of positive immune markers that precede type 1 diabetes, even in those who do not yet have altered glucose metabolism). Additionally, the present inventor, who has been a physician and clinician with special interest in type 1 type diabetes since 1984, has observed that a number of other conditions are seen among patients with type 1 diabetes including, but not limited to amyotrophic lateral sclerosis, multiple sclerosis, Parkinson's, immune system disorders that attack the basal ganglia including pediatric autoimmune neurobiological disorder, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis and Juvenile Rheumatoid Arthritis.

Unique to the art is the use of oral interferon alfa-2a at dosages 1/10,000 the dosage used in other clinical diseases in combination with an islet neogenesis or beta cell regeneration agent without the need for transplants resulting in insulin independence. Only the present inventor saw the potential for oral interferon as a BRM to be used individually or in combination with one or more beta cell regeneration and/or replacement therapies such as an islet neogenesis or beta cell regeneration agent, islet or beta cell transplants, stem cell transplants, and devices which house islets, beta or stem cells for the treatment of type 1 diabetes. Additionally, new to the art is the use of oral interferon as a preventive agent BRM for patients who have autoimmune markers for the development of diabetes.

Only the present inventor has seen the possibility of using oral interferon as part of a combination therapy with an islet neogenesis therapy, beta regeneration therapy, islet, beta cell or stem cell transplant, or a device housing islets, beta cells or stem cells implanted into a patient with type 1 diabetes and for patients at risk for developing type 1 diabetes.

The present inventor has identified that a BRM may slow the progression of loss of beta cell mass, despite lack of insulin independence among new onset type 1 diabetes children and adults, due to the findings of safety including lack of side effects compared to control group and lack of immune suppression. The present inventor has uniquely identified the potential for combining an agent, despite its lack of ability to result in insulin independence, with various therapies designed to boost or introduce new islets and/or beta cells, including islet neogenesis therapy, beta regeneration therapy, an islet, beta cell or stem cell transplant, or a device housing islets, beta cells or stem cells implanted into a patient with type 1 diabetes.

The combination of oral interferon alfa-2a with such islet and/or beta cell boosting or introducing therapies is outside the current convention, but is uniquely brought forth by the present inventor. A combination of a safe BRM, such as oral alfa-2a interferon with an islet neogenesis therapy or beta regeneration therapy, an islet, beta cell or stem cell transplant, or a device housing islets, beta cells or stem cells implanted into a patient with type 1 diabetes is new to the art.

The use of oral interferon alone or in combination with another immune agent or in combination with islet neogenesis therapy or beta regeneration therapy, with an islet, beta cell or stem cell transplant, or a device housing islets, beta cells or stem cells implanted into a patient with type 1 diabetes was not been considered by the Diabetes Immune Tolerance Network, in collaboration with the Juvenile Diabetes Research Foundation, and Type 1 Diabetes Combination Therapy Assessment Group (see Matthews et. Al., Clin Exp Immunol. 2010; 160(2):176-84).

The present inventor has identified that potential for oral interferon alfa-2a with its safety and slower decline in C-peptide among new onset patients treated with oral interferon for one year with no side effects, and only the present inventor posits combination therapies of interferon alfa-2a with one or more islet or beta cell regeneration and/or replacement therapies, including but not limited to an islet neogenesis agent, beta regeneration therapy, islet transplant, stem cell transplant or devices that house islets, beta cells or stem cells for usage in type 1 diabetes. The safety of oral interferon makes it a potential treatment of choice to protect insulin-producing cells from immune attack.

Interferon alfa was first described as a factor produced by virus infected cells in 1957 (see Isaacs et al, Proc R. Soc Lond. [Biol.] 1957, 147, 258-67). Interferon is composed of two homologous proteins (interferon alfa-2a and interferon beta). Dozens of studies in humans and animals have demonstrated the activity of oral interferon alfa-2a, which is hypothesized to work by a different mechanism orally than parentally. When administered in the morning before food, without digestive enzymes present, interferon alfa-2a is acid stable and can survive passage to the small intestine. High affinity receptors for interferon alfa-2a are found in the Peyers patches (see Brandtzaeg Curr. Topics Microbiol Immunol 1989, 146, 13-28; Mattingly, Cell Immunol 1984, 86, 46-52; Pfeffer, Cancer Res 1990, 50 2654-2657; and Pfeffer, Pharmac. Ther. 1991, 52, 149-151).

Given the importance of a therapy administered to type 1 diabetes patients which is safe and will not further compromise type 1 patients' immune systems, the present inventor sought to find a safe and effective immune agent which does not cause side-effects in children and adults, which is the basis of the present invention.

In addition to studies of oral interferon in NOD mice, interferon alfa-2a has been used in three separate human trials among new onset type 1 diabetes patients. Because of the safety that has been demonstrated with oral interferon alfa-2a and its robust biological response among patients with relapsing remitting multiple myeloma at dosages ranging from 3000 to 100,000 IU that were nontoxic, with a significant reduction in cerebral MRI enhancements in the 10,000 IU group compared to placebo, an initial study was conducted treating 10 newly diagnosed type 1 diabetes patients with 30,000 IU ingested interferon-alfa-2a (IFN-alfa-2a) within one month of diagnosis. The differences between baseline and Sustacal-induced/mixed meal C-peptide responses, respectively, at 0, 3, 6, 9, and 12 months were examined. Eight of the ten patients showed preserved beta cell function, with at least a 30% increase in stimulated C-peptide levels at 0, 3, 6, 9, and 12 months after initiation of treatment. There was no discernible chemical or clinical toxicity associated with ingested IFN-alfa-2a (see Brod et al, J Interferon Cytokine Res. 2001; 21(12):1021-30.

A randomized study was then performed among patients with recent onset type 1 diabetes (within 6 weeks of diagnosis) ages 3-25 who were randomized to receive placebo or 5000 IU or 30,000 IU oral interferon once daily for 12 months. Individuals in the placebo group (n=30) lost 56% of their C-peptide secretion Area Under the Curve (AUC) in response to a mixed meal in contrast to 29% for the 5000 IU group (n=27) and 48% for the 30,000 IU group (n=31) (p=0.028 for treatment vs. placebo groups) (see Brod et al, Cytokine Research 2001; 21.1021-1030).

A third human trial among new onset type 1 diabetes patient aged 3-25 years was conducted, with randomization to placebo (44 patients), 5000 IU of oral interferon (39 patients) or 30,000 IU oral interferon (45 patients) (see Rother et al., Diabetes Care (2009) 32; 1250-55). Patients treated with interferon has less percentage loss of mixed-meal stimulated C-peptide AUC than controls and those in the 5000 IU treated group demonstrated the preservation of beta cell mass after 12 months of treatment with a loss of 29% compared to controls with a loss of 56% of beta mass (p=0.017). The side effect profile was not different in the 5000 IU unit interferon group compared to placebo. This effect of lower dosages of oral interferon being most effective has been described in all of the studies in humans using oral interferon and has been described as the hormetic dose-response with more favorable responses to low exposures than higher exposures.

Uniquely, the present inventor redefines type 1 diabetes in man as both a disease of autoimmunity and lack of beta cell regeneration even in an immune muted milieu, which is why more than 100 human clinical trials in new onset type 1 diabetes have failed with immune agents alone and combinations of immune suppressants. The present inventor puts forth a BRM, which has not been used in combination with a islet neogenesis agent as a safe therapy to prevent beta cell loss without immune suppression.

Previous studies have shown that immune agents alone or combinations of immune therapies may temporarily slow the destruction of beta cells, but do not halt or reverse beta cell destruction. While islet transplants can result in increased insulin production, the effects are not long lasting due to the continued immune attack on new beta cells. Even successful pancreas transplants among HLA-matched identical twins require immunotherapies to preserve beta cells from immune attack, but to date, no oral BRMs have been used for islet, and beta cell or stem cell transplants for type 1 diabetes patients.

New approaches that treat both autoimmunity and aid with beta cell regeneration may hold the key to future approaches for this significant public health concern with a growing unmet medical need. To date, there have been no trials using a combination of both an effective therapy proven to increase stimulated C-peptide among type 1 patients with an agent that protects the beta cell without significant side effects or potential suppression of the immune system.

Diabetes is a chronic disease that manifests when insulin production by the beta cells of the pancreas is insufficient. Type 1 and type 2 diabetes have long been considered diseases resulting from diminished insulin secretion. Research carried out over the past century has more clearly found that generating new beta cells that make insulin is the key to reversing this disease.

Beta cells, which secrete insulin, were discovered in 1869 by a medical student, Paul Langerhans. Pancreatic islets, which are predominately comprised of beta cells are highly active metabolically, utilizing 20% of the blood supply delivered to the pancreas, but only accounting for 2% of the pancreatic mass; the remainder being extra-islet exocrine tissue containing ductal, acinar and progenitor tissue.

There is a dire need to restore new beta cells and maintain beta cell mass among type 1 diabetes patients. The loss of endogenous insulin is directly correlated with a multiplicity of atherogenic risk factors for microvascular and macrovascular complications. Lack of insulin, which is the hallmark of diabetes results not only in elevated glucose levels, but also results in a large number and wide complexity of metabolic abnormalities. For example, lack of insulin results in diminished activation of lipoprotein lipase resulting in increased levels of triglyceride-rich lipoproteins including chylomicrons and very low-density lipoproteins.

Among type 1 patients the pathology is more complicated, because despite the known autoimmune attack on beta cells, the delivery of agents to protect the beta cells from further attack has not rendered patients with sustained freedom from exogenous insulin. Despite dozens of clinical trials with a large variety and types of autoimmune therapies that were successful in reversing diabetes in NOD mice, autoimmune therapy alone provided to patients with type 1 diabetes within 3 months of their diagnosis did not sustain insulin-independence since in man, as compared to mice, there is not the significant beta cell regeneration to sustain insulin independence.

The leading hypothesis of how new beta cells can be formed in both children and adults is based upon the original works of scientists nearly a century ago who identified that in acute pancreatic injury there is new beta cell growth. Frederick Banting discovered insulin in 1921 by clamping the pancreatic ducts to induce the formation of new pancreatic cells. Dr. Banting collected the pancreatic secretions after acute pancreatic ligation and these secretions became known as insulin (see Banting F G and Best C H. J Lab Clin Med. 1922; 7:464-472). This work was supported by several earlier scientists who described that although the population of beta cells is primarily formed during embryogenesis, there is the ability to grow new beta cells post-natally through a process of transformation of ductal cell tissue into insulin-producing tissue. By 1920, the regenerative powers of the pancreas were well described. Frederick Banting attributes his studies leading to the discovery of insulin on the work of Moses Barron who documented that regeneration of injured pancreatic tissue manifests from the pancreatic ducts (see Barron M. Surg Gynec Obstet. 1920; 19:437-448). Prior to the widespread availability of insulin, surgeons performed partial pancreatectomies on diabetic children in the hopes of stimulating beta cell regeneration (see DeTakats G. Endocrinology. 1930; 14:255-264). Benefits from these novel procedures were described, but were short-lived, likely because of ongoing autoimmune destruction.

Utilizing the data available from the Human Genome Project, the present inventor and others have shown the ability to generate fully-functional pancreatic beta cells through the differentiation of non-endocrine cells. The ability of bioactive regions of the Reg gene proteins to transform extra-islet ductal tissue into islets has now been shown by more than a dozen research groups including The Section of Islet Cell and Regenerative Biology at Joslin Diabetes Center at Harvard University and The Departments of Beta Cell Regeneration at the Hagedorn Research Institute in Denmark. The Reg gene peptides identified by the present inventor and others are still in development.

The present inventor has previously shown that the human Reg gene peptides are directly involved in new beta cell formation from extra-islet ductal tissue. Others have confirmed the presence of Reg in the pancreas of newly diagnosed human diabetes, with subsequent data in both human ductal tissues and from BrdU studies showing that Reg serves to directly form new beta cells from extra-islet ductal tissue (see Levetan C S et al, Endocr Pract. (2008) 14(9):1075-1083; Rosenberg L et al, Diabetologia. (1996) 39:256-262; Li J et al, Peptides (2009) 30(12):2242-2249; and Dungan K M et al, Diabetes Metab Res Rev. (2009) 25(6):558-565).

To date the Reg peptides have emerged as possibly the most efficacious agent for regenerating new beta cells among type 1 patients, with human data demonstrating a 27% rise in arginine-stimulated C-peptide by day 54 (see Dungan K M et al, Diabetes Metab Res Rev. 2009; 25(6):558-565). Previously, the present inventor demonstrated that a human Reg3a gene protein has successfully been administered to human pancreatic ductal tissue devoid of islets resulting in a significant increase in insulin concentrations indicating new beta cell formation; a 3-fold rise in total beta cells staining insulin in STZ-rendered diabetic mice was observed (see Levetan C S., et al, Endocr Pract. 2008; 14(9):1075-1083). Reg3a protein and placebo-treated mice underwent an overnight fast and a fasting glucose level on the morning of day 39 of treatment. Fasting glucose levels were 258.00±84.5 mg/dl in the placebo group compared to a fasting glucose level of 111.00±11.4 mg/dL (p=0.020) in the Reg3a protein-treated mice.

Two studies by separate investigators have shown the ability of Reg peptide to transform human extra-islet pancreatic exocrine tissue into new beta cells in vitro. These studies were conducted by a methodology utilized in pancreatic islet transplantation in which the pancreatic endocrine beta cells are separated from the exocrine ductal tissue; the exocrine ductal tissue was shown to transform into new beta cells in the presence of Reg peptide (see Li J, et al. Peptides 2009; 30:2242-9, Assouline-Thomas B G, Diabetes 2008, 57(Suppl; 1) A2413). The current gold-standard, BrdU labeling, was used to label the beta cell lineage in rodents, which distinguishes whether new beta cells are formed by budding from pre-existing beta cells versus being formed from extra-islet ductal exocrine tissue (see Kapur R, et al, Islets. 2012; 4(1)).

The Section of Islet Cell and Regenerative Biology at Joslin Diabetes Center found that the 15-amino acid hamster INGAP Reg3 gamma peptide was present in the newest beta cells and islets that were formed directly from branching proliferating extra-islet ducts, which also confirms that the mechanism of action of Reg peptide is to form new beta cells from extra-islet exocrine tissue (see Guo L et al, Diabetes. 2010, 59 (suppl; 1) A2589). When Reg is inhibited by the administration of a blocking antibody in an animal model of pancreatic injury there was attenuated recovery, also confirming that Reg's role is both protective and regenerative during acute pancreatic injury (see Viterbo D, et al. JOP. 2009; 10(1):15-23).

The Departments of Beta Cell Regeneration at the Hagedorn Research Institute and Peptide and Protein Chemistry at Novo Nordisk reported a 2-fold increase in the volume of new small islets developing from non-endocrine tissue resulting from the treatment with both the human 14 amino acid Reg3a peptide, HIP, and the 15-amino acid Reg3gamma hamster peptide, INGAP (see Kapur R, et al, Islets. 2012; 4(1)). Five days after treatment with both the 14-amino acid human Reg3a peptide, HIP, and the 15-amino acid hamster Reg3gamma peptide, INGAP, increased levels of new islet markers necessary for islet formation were observed, including NGN3, NKX6.1, SOX9, and INS, indicating that REG is a catalyst for beta cell neogenesis (see Kapur R, et al, Islets. 2012; 4(1). Similar to these findings, other data support that the Reg protein is an initiating factor for downstream regulation of new beta cells (see Levetan C., 2010, J Diabetes; 2(2):76-84). For example, when Reg is initially expressed, PDX-1, PAX1, Ngn3, Nkx6.1, Sox9, and Ins are not expressed; once Reg is present, PDX-1, PAX1, Ngn3, Nkx6.1, Sox9 and Ins and other beta cell proliferation factors become present demonstrating that Reg activates downstream factors necessary for beta cell regeneration (see Vukkadapu S S. Physiol Genomics 2005:21, 201-211 and Kapur R., et al., Islets. 2012; 4(1)). Gurr and colleagues confirmed positive Reg staining in ductal epithelium in acutely diabetic NOD mice and in the pancreas of a type 1 healthy cadaveric human pancreata or in healthy mice (see Gurr. Diabetes 2002. 51(2):339-346)

The organ specificity of Reg proteins to the pancreatic ducts has been illustrated by tagged Reg protein labeled with fluorescein isothiocyanate that was administered via intraperitoneal injection to rodents. The only organ that had fluorescent staining was the pancreas with labeling only found specifically within the nonendocrine pancreatic ductal populations, again confirming that the mechanism of action of Reg is transformation of extra-islet ductal cells into beta cells (see Pittenger G L et al, Diabetologia 2009; 52 (5):735-738). There are now numerous studies confirming that the mechanism of action of the Reg peptides is to transform extra-islet exocrine ductal tissue into new islets rather than the newly formed beta cells resulting from the budding from existing beta cells.

The present inventor has also investigated the role and pathways of other human hormones involved in beta cell regeneration with findings consistent with initial findings of Moore and colleagues in 1906, demonstrating the role of gastrointestinal hormones in improving diabetes control among three patients with type 1 diabetes (see Levetan C. 2010, J Diabetes; 2(2):76-84; Moore et al, Biochem J. 1906; 1(1): 28-38). The mechanism of action of these gastrointestinal hormones were not only found to be in insulin secretion, but decades later these gut peptides have been shown to be involved in the transformation of extra-islet exocrine tissue into new endocrine tissue containing beta cells (see Wang T C. J Clin Invest. 1993; 92(3):1349-56).

Not until 1999, when the use of cell lineage labeling became available, did the embryological concepts of the pancreas change. Whereas it had been thought that the pancreas was derived from both ectoderm and endoderm, it has now been shown that the entire pancreas arises only from endoderm during embryological development. This helps explain how beta progenitor cells have been described as residing diffusely throughout the adult pancreatic tissue and how growth factors transform pancreatic extra-islet ductal tissue into new beta cells. Over the past several decades, the ability to regenerate new beta cells from progenitor cells found within the pancreatic ductal tissue has been illustrated by many teams.

Despite many trials with numerous immune agents among type 1 patients and those at risk for type 1 diabetes, it is the present inventor who first hypothesized that an immune protectant would only be effective with an islet neogenesis agent. The present inventor hypothesizes the use of a safe BRM that does not suppress the immune system while protecting newly formed beta cells with an islet neogenesis, beta regeneration agent or other methods of delivering islets, beta cells and stem cells and it is this combination of providing a protector of beta cells in combination with new beta cells to patients with diabetes that is critical for insulin independence.

To date, there have been no studies that combine an oral BRM with a known islet neogenesis therapy. The prior art in the field has described the usage of gastrin with other growth factors, but has never specifically used a BRM in combination with these agents (see U.S. Pat. No. 6,992,060).

The present inventor has shown great distinctions between the insulin-producing islets of mice and men with humans having much more complex islet structures with respect to composition of cell type, neural and vascular innervation and unique paracrine interactions that are not found in rodents. Levetan has demonstrated vast differences in the islets of mice and men, which may explain the many, many studies conducted among rodent models in the field of diabetes that later are unable to be replicated in human studies (see Levetan C S et al. Endocr Pract. 2012; 27:1-36). Specifically, trials with multiple different agents and types of agents have been utilized in preclinical rodent models evaluating agents that may be successful in clinical practice for usage in patients with type 1 diabetes. The present inventor has also previously shown, like many other scientific teams, that after fetal development of beta cells, typically new beta cells are only derived from the existing, surviving beta cell population. Different and unique to the previous art in the field, the present inventor has shown the ability to postnatally generate new beta cells by the transformation of human pancreatic ductal tissue (see Levetan C. J Diabetes. 2010; 2(2):76-84; Levetan C S. Endocr Pract. 2008; 14(9):1075-83).

New and unique research by the present inventor, which has not been obvious in the prior art, is 1) the ability to reverse diabetes in the diabetic mouse models may be flawed by the complexity of the human islet compared to that of the rodent and 2) the process of generating new beta cells must be from a different source than from the beta cells remaining after the diagnosis of type 1 or type 2 diabetes is made because of the limited supply (<10% for type 1 diabetes and <50-75% for type 2 diabetes). The present inventor has shown the ability to transform new pools of beta cells within new islets from extra-islet ductal tissue, which contain progenitor cells, which can form new islets (see U.S. Pat. Nos. 8,211,430; 7,989,415; 7,714,103; and 7,393,919).

There is a need in the art for new therapeutic modalities for the treatment of diabetes in humans that generate new beta cells from extra-islet tissue while preserving the population of nascent beta cells from destruction by the immune system using an oral BRM rather than an immune suppressant therapy.

### BRIEF SUMMARY OF THE INVENTION

Embodiments of the present invention provide for novel therapies, pharmaceutical compositions and methods for insulin independence utilizing an oral Biological Response Modifier (BRM). As used herein, a Biological Response Modifier (BRM) is defined as a therapy that modifies the immune system's attack on insulin-producing beta cells, but does not suppress the body's immune system and is not associated with adverse effects beyond that of a placebo. The BRM includes interferon alfa-2a used orally at a fraction of the doses used to treat Hairy Cell Leukemia, Multiple Sclerosis and Hepatitis C. For example, 5000 IU interferon alfa-2a is 1/10,000 of the subcutaneous dosage for treating these conditions. Embodiments of the invention provide interferon alfa-2a or pegylated interferon alfa-2a at a dose of 1 IU to 50,000 IU, including 1, 10, 25, 50, 75, 100, 200, 300, 400, 500, 600, 700, 800, 900, 1000, 1250, 1500, 1750, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000 IU, or any range encompassing or including these values such as 4000 to 6000 IU, 3000 to 7000 IU, 2000 to 8000 IU, 2500 to 7500 IU, 1000 to 10000 IU, 2500 to 10000 IU, 1000 to 25000 IU, 1000 to 30000 IU, 1000 to 40000 IU, 1000 to 50000 IU, and so on. In some embodiments, interferon alfa-2a or pegylated interferon alfa-2a is provided at a dose of about 5000 IU.

In embodiments, the interferon alfa-2A is used a BRM for protection of insulin-producing beta cells against immune attack in those at risk for development of type 1 diabetes to prevent or delay the onset of type 1 diabetes. In other embodiments, interferon alfa-2A is used prior to development of any alteration in glucose metabolism. In other embodiments, interferon alfa-2A is used in those already diagnosed with type 1 diabetes in conjunction with islet neogenesis therapy or beta regeneration therapy, with an islet, beta cell or stem cell transplant, or in combination with an implanted device housing islets, beta cells or stem cells. In other embodiments, interferon alfa-2A is used to treat a number of conditions found in patients with type 1 diabetes and their relatives, for which current therapy or for which therapy is no longer effective in the progression of the disease, which includes, but is not limited to amyotrophic lateral sclerosis and multiple sclerosis and may be used for other autoimmune disorders that have no treatment including forms of Parkinson's disease, immune system disorders that attack the basal ganglia including pediatric autoimmune neurobiological disorders associated with streptococci, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis, Juvenile Rheumatoid Arthritis.

Oral interferon alfa-2a has never been considered in the prior art to be used alone or in combination with other immune agents and specifically not considered to be an option for type 1 diabetes as stated by the Section Chief, at the National Institutes of Health, of Pediatric Diabetes and Metabolism, Diabetes, Endocrinology, and Obesity Branch Diabetes Immune Tolerance Network, Juvenile Diabetes Research Foundation, and Type 1 Diabetes Combination Therapy Assessment Group. Methods, pharmaceutical compositions and therapies novel to the prior art based on the use of an oral BRM for protection of beta cells against autoimmune attack are utilized to render patients with recent onset and existing type 1 diabetes, insulin independent by utilizing one or more of an islet or beta regeneration therapy, islet, beta cell or stem cell transplant or implanted devices housing islets, beta cells or stem cells in combination with the BRM.

In addition, embodiments of the invention include the use of oral interferon for prevention of type 1 patients in those at risk who may have no glucose impairment, but have first or 2^{nd} degree relatives with the disease and possess antibody markers associated with the development of the disease. There have been more than 163 human clinical trials to prevent type 1 diabetes using compounds such as allopurinol, CTLA4-Ig (Abatacept), Sirolimus (Rapamycin), Tacrolimus (FK506), Etanercept, Alefacept, Belatacept, a heat-shock protein 60 (Diapep277), a tuberculosis vaccine, Glutamic Acid Decarboxylase 65 (GAD65) vaccine, the BCG tuberculosis vaccine also known as Bacillus Calmette-Guérin or Bacille Calmette-Guérin/BCG Vaccine, Mycophenolate Mofetil alone or in combination with Daclizumab, the anti-CD20 agent Rituximab; Campath-1H (Anti-CD52 Antibody), lysofylline, antithymocyte globulin (ATG), Proleukin and those the combination of Proleukin and Rapamune, Vitamin D (Vitamin D2, D3, 1.25 dihydroxy D and other Vitamin D preparations), IBC-VSO vaccine, Ex vivo Expanded Human Autologous CD4+CD127lo/−CD25+ Polyclonal Regulatory T Cells, a vaccine using CD4⁺CD25⁺ antigen-specific regulatory T cells, Interleukin-1 Receptor Antagonist (anakinra), and Alfa-2a 1-Antitrypsin and others. None have exhibited efficacy alone or resulted in insulin independence among type 1 patients.

The present inventor provides data to suggest that oral interferon, when given at the earliest time or recognition of the possibility of type 1 diabetes, is a safe and effective therapy that may prevent the onset of type 1 diabetes in those identified as being at risk for the disease by expressing antibodies associated with type 1 diabetes or having relatives with type 1 diabetes, knowing that there is a window as long as 10 years between antibody expression and development of abnormal glucose metabolism. The present inventor provides a safe BRM that has not been shown to have side effects beyond that of placebo in randomized trials among children and adults aged 3-25 years old and that has preserved beta cell mass in these patients. Additionally, oral interferon alfa-2a has been shown to be effective in NOD mice and in slowing the loss of beta cells in three human trials among new onset type 1 children and adults.

While not wishing to be bound by theory, embodiments of the invention administer oral interferon alfa-2a in oral dosages of about 2500 to 7500 IU per day in combination with an islet neogenesis agent, beta regeneration agent, stem cell, beta cell or islet transplant or with any device housing beta cells, islets or stem cells or any therapy which increases beta cell mass, such that oral interferon alfa-2a may diminish the attack on new and existing beta cells and may be used with any other described immune therapies.

Further, it has been shown that peptides patented by the present inventor have been shown to transform pancreatic ductal tissue to islet using the gold standard BrDU method with upregulation of transcription factors using the Reg peptides patented by the present inventor (see Kapur R. Islets. 2012 January-February; 4(1):40-8.)

Embodiments of the invention identify for the first time a combination of therapies that includes one or more islet and/or beta cell regeneration or replacement therapies such as islet and beta regeneration agents, beta cell, islet or stem cell transplants, or devices housing islets, beta cells with a safe oral BRM, such as oral interferon alfa-2a or PEGylated oral interferon alfa-2a for the protection of new beta cells generated by such therapies for patients with type 1 diabetes. The therapeutic methods described in this invention are not described in the prior art, and specifically include, but are not limited to, oral interferon alfa-2a as a BRM.

The present inventor has shown great distinctions between the insulin-producing islets of mice and men with humans having much more complex islet structures with respect to composition of cell type, neural and vascular innervation and unique paracrine interactions that are not found in rodents, and understands why immune suppressant drugs alone do not work in humans and that a unique approach using an oral BRM with an islet and/or beta cell therapy may uniquely result in insulin independence without the need for a therapy that suppresses the immune system.

Embodiments of the invention provide a new model for treatment of type 1 diabetes. Based upon the complexity and distinctions between the islets of mice and men, embodiments provide for novel therapies, pharmaceutical compositions and methods for insulin independence and provide a methodology for treating patients requiring insulin that have not previously been described. Such embodiments include compositions of an oral BRM or method of treating or preventing type I diabetes using an oral BRM in combination with one or more islet and/or beta cell regeneration or replacement therapies such as a beta regeneration therapy, transplants of islets, beta cells or stem cells, or devices implanted in type 1 patients containing islets, beta cells, or stem cells.

Additional embodiments include in vivo methods for treating or preventing type I diabetes or associated conditions which use a combination of therapies, which combination includes an oral BRM such as oral interferon alfa-2a. Embodiments also include methods of ex vivo transformation of extra-islet ductal cells or pluripotent stem cells into new beta cells, which are then administered to patients with new and existing type 1, or diseases of insulin deficiency, beta cell deficiency, insulin resistance and impaired glucose metabolism in combination with administration of an oral BRM.

Other embodiments include methods for pancreatic beta cell generation and include both in vivo and ex vivo beta cell generation and methods for treating new onset and previously existing type 1 diabetes, Latent Autoimmune Diabetes of Adulthood (LADA), and those at risk for type 1 diabetes, including but not limited to those with positive autoimmune antibodies markers including insulin, IA2, ZnT8 or Glutamic Acid Decarboxylase-65 antibody, ZnT8 or any markers of autoimmune diabetes or autoimmune Pre-Diabetes or diseases of hyperglycemia, glucose intolerance and beta cell impairment or deficiency associated with autoimmunity.

Additionally, based on efficacy of oral interferon alfa-2a in maintaining beta cell mass significantly beyond that of placebo in children and adults, embodiments include methods to prevent or delay type 1 diabetes in those at risk for the disease, patients with diabetes, or family members who may have other associated autoimmune conditions and in which an autoimmune basis is hypothesized for which there may be no successful treatment including, but not limited to amyotrophic lateral sclerosis and multiple sclerosis, forms of Parkinson's, immune system disorders that attack the basal ganglia including pediatric autoimmune neurobiological disorders, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis, and Juvenile Rheumatoid Arthritis.

Embodiments of the invention describes the usage of oral BRMs, including but not limited to oral interferon alfa-2a or PEGylated alfa-2a in combination with agents or therapies that result in new beta cell formation including, but not limited to Reg peptides, derivatives, optimized forms including peptidomimetics of the Reg peptides and stimulating antibodies to the Reg receptor and other novel agents for beta cell generation, beta regeneration agents, islet, beta or stem cell transplants or implanted devices housing islet, beta or stem cells. Previous work by the present inventor and others has shown the potential for in vivo and ex vivo transformation of human extra-pancreatic ductal tissue into new islets, which contains new beta cell populations (see U.S. Pat. Nos. 8,211,430; 7,989,415; 7,714,103; and 7,393,919).

Embodiment of the invention also provides for pharmaceutical compositions comprising BRMs with beta regeneration and islet agent(s), peptidomimetics formed to bind with the Reg receptor binding region or stimulatory antibodies to the Reg receptor binding region resulting in islet neogenesis, as well as kits comprising the same.

### BRIEF DESCRIPTION OF THE SEQUENCES

SEQ ID NO:1 is an embodiment of a peptide of the present invention, which is 9 amino acids in length. SEQ ID NO:1 is a partial sequence of human Regenerating islet-derived 1alpha (Reg1a).

SEQ ID NO:2 is human Regenerating islet-derived 1alpha (Reg1a), also known as human lithostathine-1-alpha precursor (public accession number NP_002900)

SEQ ID NO:3 is human Regenerating islet-derived 1beta (Reg1b), also known as lithostathine-1-beta precursor (public accession number NP_006498).

SEQ ID NO:4 is an embodiment of a peptide of the present invention, which is 8 amino acids in length. SEQ ID NO:4 is a partial sequence of human Reg1a, human Reg1b, and human Reg3a.

SEQ ID NO:5 is human regenerating islet-derived protein 3-alpha precursor (Reg3a) (public accession number NP_002571).

SEQ ID NO:6 is the human Reg receptor, also known as exostosin-like 3 (public accession number NP_001431).

SEQ ID NO:7 is an embodiment of a peptide of the present invention, which is 7 amino acids in length. SEQ ID NO:7 is a partial sequence of human Reg1a, human Reg1b, human Reg3a, and human Reg4.

SEQ ID NO:8 is an embodiment of a peptide of the present invention, which is 8 amino acids in length. SEQ ID NO:8 is a partial sequence of human Reg3a.

SEQ ID NO:9 is an embodiment of a binding site within the human Reg receptor for peptides of the present invention. SEQ ID NO:9 is a partial sequence of the human Reg receptor.

SEQ ID NO:10 is human regenerating islet-derived protein 4 isoform 1 precursor (Reg4) (public accession number NP_114433).

SEQ ID NO:11 is a 14-amino Reg3a peptide sequence (Human Proislet Peptide (HIP)) (SEQ ID NO: 3 of U.S. Pat. No. 7,393,919).

SEQ ID NO:12 is a 15-amino acid peptide within the hamster Reg3gamma peptide sequence (Islet Neogenesis Associated Protein (INGAP)) (amino acid residues 103 to 117 of SEQ ID NO: 2 of U.S. Pat. No. 5,834,590).

SEQ ID NO:13 is an N-terminal partial sequence of the human Reg receptor.

SEQ ID NO:14 is a 9-amino acid peptide within human Reg3a.

SEQ ID NO:15 is the 15-amino acid peptide within the hamster Reg3gamma peptide sequence (Islet Neogenesis Associated Protein (INGAP)) that has been blocked with a n-terminal acetyl group and an c-terminal amide group.

SEQ ID NO:16 is the 15 amino acid hamster Reg3gamma peptide within the hamster Reg3gamma peptide sequence (Islet Neogenesis Associated Protein (INGAP)) that has an additional n-terminal cysteine residue.

SEQ ID: NO:17 is the 15 amino acid Reg3gamma hamster peptide within the hamster Reg3gamma peptide sequence (Islet Neogenesis Associated Protein (INGAP)) in dimeric form, wherein each monomer has been modified to include an n-terminal cysteine residue. The dimer forms via the creation of a disulfide bond between the cysteine residues of the individual monomers.

SEQ ID NO:18 is the 15 amino acid Reg3gamma hamster peptide within the hamster Reg3gamma peptide sequence (Islet Neogenesis Associated Protein (INGAP)) in dimeric form, wherein each monomer has been modified to include an n-terminal cysteine residue and has been blocked with an n-terminal acetyl group and a c-terminal amide group. The dimer forms via the creation of a disulfide bond between the cysteine residues of the individual monomers.

SEQ ID NO:19 the 15 amino acid Reg3gamma hamster peptide within the hamster Reg3gamma peptide sequence (Islet Neogenesis Associated Protein (INGAP)), which has been modified to include an n-terminal cysteine residue to which has been covalently bonded to a dimeric maleimide activated 40 Kd PEG construct.

SEQ ID NO:20 is the 15 amino acid Reg3gamma hamster peptide within the hamster Reg3 gamma peptide sequence (Islet Neogenesis Associated Protein (INGAP)), which has been blocked with an n-terminal acetyl group and a c-terminal amide group, and modified to include an n-terminal cysteine residue to which has been covalently bonded to a dimeric maleimide activated 40 Kd PEG construct.

SEQ ID NO:21 is the 15 amino acid Reg3gamma hamster peptide within the hamster Reg3gamma peptide sequence (Islet Neogenesis Associated Protein (INGAP)) that has been blocked with an n-terminal acetyl group and a c-terminal amide group and pegylated at the C-terminus.

SEQ ID NO:22 is the 15 amino acid Reg3gamma hamster peptide within the hamster Reg3gamma peptide sequence (Islet Neogenesis Associated Protein (INGAP)) which has been blocked with a c-terminal amide group and pegylated at the n-terminus.

SEQ ID NO:23 is a 17 amino acid Reg peptide sequence which has been cyclized by way of a cyclic amide bond between side chain of Asp on position 1 and Lys on position 17 and which has been blocked with an n-terminal acetyl group and a c-terminal amide group.

SEQ ID NO:24 is the 14 amino acid human Reg3a peptide (Human Proislet Peptide (HIP)) that is blocked with an n-terminal acetyl group and a c-terminal amide group.

SEQ ID NO:25 is the 14 amino acid human Reg3a peptide (Human Proislet Peptide (HIP)) which is blocked with an n-terminal acetyl group and a c-terminal amide group and pegylated at the c-terminus.

SEQ ID NO:26 is the 14 amino acid human Reg3a peptide (Human Proislet Peptide (HIP)) which is blocked with a c-terminal amide group and pegylated at the n-terminus.

SEQ ID NO:27 is a 16 amino acid Reg peptide which has been cyclized by way of a cyclic amide bond between side chain of Asp on position 1 and Lys on position 16 and which has been blocked with an n-terminal acetyl group and a c-terminal amide group.

SEQ ID NO:28 is a 7-amino acid Reg peptide that is blocked with an n-terminal acetyl group and a c-terminal amide group.

SEQ ID NO:29 is a 7-amino acid Reg peptide that is blocked with an n-terminal acetyl group and a c-terminal amide group and pegylated at the C-terminus.

SEQ ID NO:30 is a 7-amino acid Reg peptide which is blocked with a c-terminal amide group and pegylated at the n-terminus.

SEQ ID NO:31 is a 9-amino acid Reg peptide which has been cyclized by way of a cyclic amide bond between the side chain of Asp on position 1 and Lys on position 9 and which has been blocked with an n-terminal acetyl group and a c-terminal amide group.

SEQ ID NO:32 is a 8-amino acid Reg peptide that has been blocked with an n-terminal acetyl group and a c-terminal amide group.

SEQ ID NO:33 is a 8 amino acid Reg peptide that is blocked with an n-terminal acetyl group and a c-terminal amide group and pegylated at the C-terminus.

SEQ ID NO:34 is a 8 amino acid Reg peptide which is blocked with a c-terminal amide group and pegylated at the n-terminus.

SEQ ID NO:35 is a 10 amino acid Reg peptide which has been cyclized by way of a cyclic amide bond between side chain of Asp on position 1 and Lys on position 10 and which has been blocked with an n-terminal acetyl group and a c-terminal amide group.

SEQ ID NO:36 a 9 amino acid Reg peptide which has been blocked with an n-terminal acetyl group and a c-terminal amide group.

SEQ ID NO:37 is a 9 amino acid Reg peptide that is blocked with an n-terminal acetyl group and a c-terminal amide group and pegylated at the C-terminus.

SEQ ID NO:38 is a 9 amino acid Reg peptide which is blocked with a c-terminal amide group and pegylated at the n-terminus.

SEQ ID NO:39 is an 11 amino acid Reg peptide which has been cyclized by way of a cyclic amide bond between the side chain of Asp on position 1 and Lys on position 11 and which has been blocked with an n-terminal acetyl group and a c-terminal amide group.

SEQ ID NO:40 is the 165 amino acid recombinant interferon alfa-2A polypeptide and has public accession number 1ITF_A.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

Additionally, the patent application file contains at least one drawing executed in color. Copies of this patent or patent application publication with color drawing(s) will be provided by the Office upon request and payment of the necessary fee.

FIG. 1A is a microscopic image of a human islet.

FIG. 1B is a microscopic image of a mouse islet.

FIG. 2 is an image of the tertiary protein structure of interferon alfa-2a.

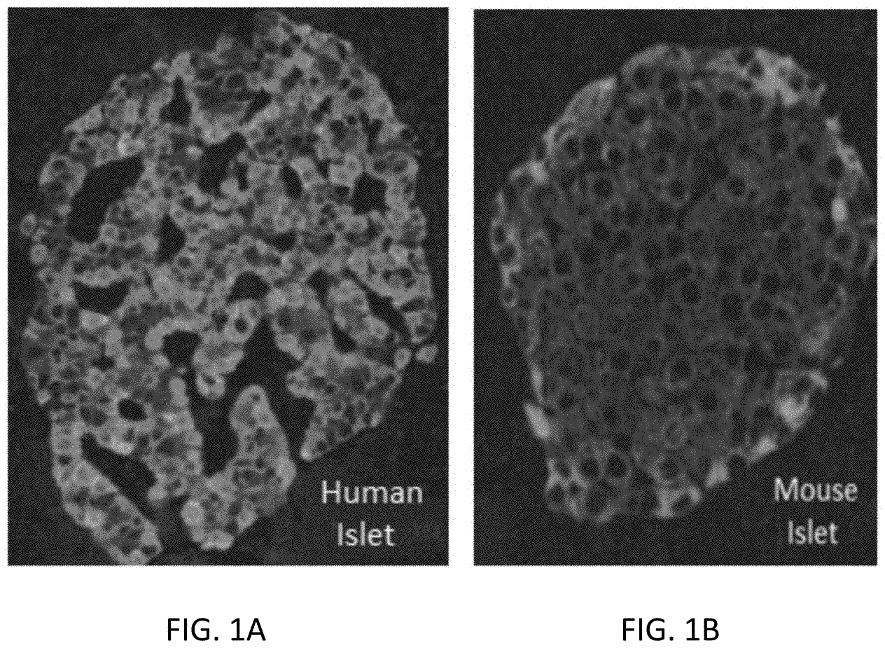

### DEFINITIONS

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of diabetes, diminishment of extent of disease, delay, slowing, or prevention of disease progression, amelioration, palliation or stabilization of the disease state, and other beneficial results described below. Symptoms of type 1 diabetes include low or inadequate levels of insulin or insulin activity, frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections, hyperglycemia, loss of glycemic control, fluctuations in postprandial blood glucose, fluctuations in blood glucagon, fluctuations in blood triglycerides. Diabetes may be diagnosed by methods well known to one of ordinary skill in the art. For example, commonly, diabetics have a plasma blood glucose result of greater than 126 mg/dL of glucose. Pre type 1 diabetes, which may also be treated by the compositions and methods of the invention is commonly diagnosed by autoimmune antibodies (GAD65, insulin, IA-2 and ZnT8) found in the blood of family members who have type 1 diabetes.

As used herein, “reduction” of a symptom or symptoms (and grammatical equivalents of this phrase) means decreasing of the severity or frequency of the symptom(s), or elimination of the symptom(s).

As used herein, “impaired glucose homeostasis” is a diminished capacity in a subject for regulating glucose by a system of feedback controls, so as to stabilize health and functioning. Conditions that are associated with or are a risk factor for impaired glucose homeostasis include new onset type 1, previously existing type 1 and 2 diabetes with unusual characteristics or poor response to medication that may have positive autoimmune antibodies for diabetes, latent autoimmune diabetes of adulthood (LADA), glutamic acid decarboxylase-65 (GAD65), insulin, IA-2 and ZnT8 autoimmunity, any condition in which a family member of a patient with type 1 diabetes has GAD65, insulin, IA-2 and ZnT8 antibodies or any antibodies that are markers for the potential of type 1 diabetes in the future or any forms of diabetes which does not respond to oral diabetic agents or non-insulin injectables or any case in which a patient is unresponsive to traditional medications including insulin and the usage of oral interferon alfa-2a is helpful for the patient when given with an islet neogenesis agent, beta regeneration agent, islet, beta or stem cell transplant or device containing islets, stem cells or receipt of an implantable device containing

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “subject” or “patient” is a mammal, typically a human, but optionally a mammalian animal of veterinary importance, including but not limited to horses, cattle, sheep, dogs, and cats. “Patient” and “subject” may be used interchangeably herein.

As used herein, a “therapeutically effective amount” of a drug or agent is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “therapeutically effective amount” of a drug may also be an amount of a drug that when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a therapeutically effective amount may be administered in one or more administrations.

As used herein, an “effective amount” of a drug or agent (and grammatical equivalents of this phrase, e.g. “amount of X that is effective”) is an amount of a drug or agent that will have the intended pharmacological or pharmacodynamic effect. The “effective amount” may apply to in vivo, in vitro, or ex vivo applications of the drug or agent.

As used herein in vitro is in cell culture, ex vivo is a cell that has been removed from the body of a subject and in vivo is within the body of a subject.

Abbreviations used herein include BRM for a biologic response modifier.

Interferon alfa-2A is known in the art in its recombinant and pegylated forms.

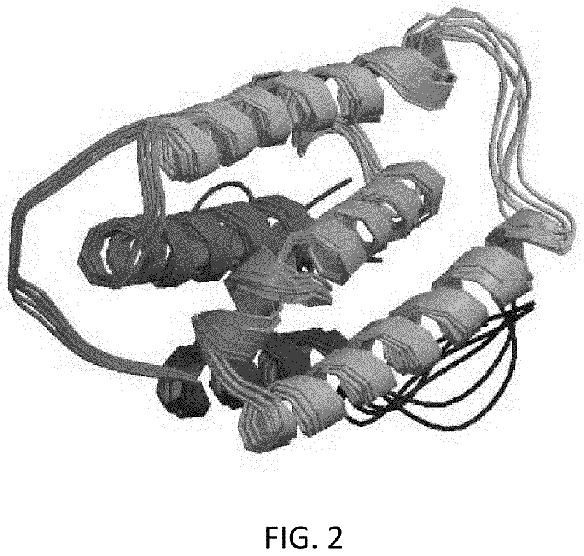

Recombinant interferon alfa-2A is a polypeptide containing 165 amino acids and has a molecular mass of 19241 Daltons. The sequence is provided in SEQ ID NO:40 and has public accession number 1ITF_A. The tertiary protein structure is provided in FIG. 2.

PEGylated interferon alfa-2a (PEGylated with a branched 40 kDa PEG chain) with synonyms including rHUPEG-IFN-alfa-2a, rHuPEG-IFN-a 2a, rHUPEG-IFN-alfa-2a, rHUPEGr-INFalfa-2a, PEGylated Interferon-alfa-2a, PEGylated Inteferonalfa-2a, PEGylated Interferon-a 2a, PEGylated Interferon-alfa-2a, and Pegasys has CAS Registry No. 198153-51-4 and has an approximate molecular weight of 60,000 daltons.

### DETAILED DESCRIPTION OF THE INVENTION

Embodiments of the present invention relate to novel therapies, pharmaceutical compositions and methods for insulin independence in patients with type 1 diabetes utilizing a BRM in combination with one or more therapies that provide a patient with type 1 diabetes new insulin-producing cells, which may include islet and beta regeneration therapies, including those have been patented by the present inventor, islet, beta cell or stem cell transplantation, or implanted devices housing islets, beta cells or stem cells. Such a combination has never previously been considered for use together for the treatment of type 1 diabetes other than by the present inventor. Additionally, embodiments include the prevention and treatment of autoimmune conditions such as neurological conditions for which there are no therapies available using oral interferon alfa-2a.

The novel methods, pharmaceutical compositions and therapies are utilized to render patients with recent onset and existing type 1 diabetes, insulin independent by utilizing a BRM with one or more islet and/or beta cell regeneration or replacement therapies, such as the Reg peptides that have been patented by the present inventor, islet, beta cell or stem cell transplantation, or implantation of devices housing islets, beta cells or stems to patients with type 1 diabetes. Not wishing to be bound by theory, BRMs protect beta cell mass from autoimmune destruction without immune suppression or side effects, and alone have not resulted in insulin independence, but may result in insulin independence in patients with type 1 diabetes when combined with islet and/or beta regeneration therapies, such as the ones that have been patented by the present inventor, or islet, beta cell or stem cell transplantation or implanted devices housing islets, beta cells or stem cells.

In embodiments, among patients with type 1 diabetes, islet or beta regeneration agents, islet, beta or stem cell transplants, and/or devices that encapsulate islets, beta cells or stem cells must be used in combination with a BRM to protect the new beta cells from autoimmune attack and therefore together there is the ability to generate new and protected beta cells that render patients with type 1 diabetes are rendered insulin-free. Embodiments of the invention provide new and unique methods, therapeutics and pharmaceutical compositions for insulin-independence among patients with type 1 diabetes. Such embodiments provide improvements to the art by providing for the usage of BRMs, in combination with therapeutics which augment and/or replace islet and/or beta cells such as regeneration agents (such as Reg peptides), islet transplants, beta cell transplants or stem cell transplants, or devices that house islets, beta cells or stem cells, to allow for insulin-independence among patients with type 1 diabetes.

Reference will now be made in detail to various exemplary embodiments of the invention. The true scope of the invention is defined by the claims. Further, any features of any embodiment described herein are equally applicable to any other embodiment described herein or envisioned by one of ordinary skill in the art. The detailed description provided herein should not be construed to exclude features otherwise described with respect to another embodiment.

In one embodiment, the present invention provides a method of treating recent onset, existing type 1 diabetes and Latent Autoimmune Diabetes of Adulthood (LADA) by administration of a BRM, not limited to oral interferon alfa-2a or PEGylated oral interferon alfa-2a, in combination with an islet or beta regeneration agent (such as those patented by the present inventor) or with an islet, beta cell or stem cell transplant or a device that encapsulates beta cells, islets or stem cells for the purpose of providing insulin and glucose homeostasis. Not wishing to be bound by theory, the use of a BRM may generate immune protection of beta cells, while islet regeneration and beta regeneration agents as patented by the present inventor transform pancreatic extra-islet ductal tissue into new beta cells and confers specific regenerative capacity on the human pancreas. The combination of a BRM with a therapy that provides insulin to patients with type 1 may reduce or eliminate the need for exogenous insulin dependence in these patients.

The BRM may also be used in combination with other beta regeneration agents including, but not limited to Reg Peptides, Optimized Reg Peptide formulations and/or agents that bind to the human Reg Receptor.

Exemplary oral BRMs that may be used in the invention include, but are not limited to oral usage of interferon alfa-2a and oral PEGylated interferon alfa-2a.

In one embodiment, methods for treating a pathology associated specifically with impaired pancreatic function in a subject with type 1 diabetes are provided. The method comprises the steps of administering an islet neogenesis agent (such as an optimized 14 or 15 amino acid Reg peptide) in a dosage ranging from 60-300 mg subcutaneously daily, such as 60, 70, 80, 90, 100, 110, 120, 130, 140, 150, 160, 170, 180, 190, 200, 210, 220, 230, 240, 250, 260, 270, 280, 290, or 300 mg, or at any range encompassing or including these values. In other embodiments, the islet neogenesis agent may be delivered orally or given via in a slow-release delivery system that is implanted in the patient with diabetes. The islet neogenesis agent is administered in combination with a daily oral dosage of about 5000 IU of oral interferon alfa-2a taken to prevent autoimmune attack on newly formed beta cells as formed by Reg peptide or by the implanted islets, beta cells or stem cells.

The method may further comprise one or more of the steps of (1) intensifying glycemic control (2) administering oral vitamin D to maintain 25-hydroxyvitamin levels above 40 mg/ml in order for optimizing islet neogenesis using a Reg peptide and using oral interferon to protect new beta cells within newly formed and existing beta cells

During the process of in-vivo new beta cell formation from extra-islet ductal tissue, it is critical that glucose levels remain within a narrow range. Because of the many redundant mechanisms in the body to prevent hypoglycemia including the secretion of epinephrine, norepinephrine, cortisol and growth hormone to protect against hypoglycemia, hypoglycemia is a contraindication for growth of new beta cells. Similarly, if glucose levels are markedly elevated, there is glucose toxicity to beta cells, thus beta regeneration in the presence of a beta promoting agent like a islet neogenesis therapy beta regeneration agent or stem cells is best given with a meal when there will be a peak post-meal glucose level. Thus, the islet neogenesis therapy may be dosed with breakfast and dinner with about 5000 IU of oral interferon alfa-2a being given in the morning.

Significant hypoglycemia in patients would not be optimal for islet neogenesis. Compared to rodents, glucose levels are slightly lower in humans. Despite this difference, homeostasis is maintained within a very narrow range in both species, due to the exquisite intercommunication within the islet complex. Sensor data from non-diabetic humans demonstrate that 80% of all measured glucose levels lie within 60-100 mg/dL, with mean peak glucose levels after meals of <120 mg/dL. Linear regression curves from the Diabetes Control and Complications Trial (DCCT) and the United Kingdom Prospective Diabetes Study (UKPDS) show that A1C levels above 5.5% are associated with more complications. This data is supported by A1C levels from the EPIC-Norfolk trial among non-diabetic individuals, which found that A1C levels above 5.5% are associated with significantly increased risks for vascular-related morbidity and mortality.

Glucose homeostasis requires an adequate number of completely functional islets, as illustrated by the inability to restore normoglycemia among diabetic patients even when intensive regimens of insulins are utilized. The DCCT investigators set, as a major treatment outcome goal, a mean A1C over the trial period of ≤6.05% without an increased risk for hypoglycemia This goal was not achieved by only replacing insulin, that is, only one of the multiple hormones missing in diabetes. The relationship between distinct cell types within the islet and the accompanied islet abnormalities in resulting from beta cell loss, including dysfunction with amylin, glucagon, somatostatin, pancreatic polypeptide and islet ghrelin had yet to be and continues to be elucidated.

Sensor-augmented pumps recently were shown to improve A1C levels from 8.3% to 7.5% over 12 months, with further reductions to 7.4% after an additional 6 months of treatment. These achievements were made without the associated weight gain or hypoglycemia seen in the DCCT. Despite technological advances in sensors and pumps, sensor-augmented pump therapy did not improve A1C levels as much as those seen in the DCCT decades ago. This underscores the importance of restoring beta function and communication within the islet complex.

For example, a clinical study would use both an agent that regenerates islets and a therapy that protects beta cells from autoimmune destruction. The glucose goals would be 100 mg/dL range before meals and 140 mg/dL two hours after meals. Once patients are enrolled in such a trial, patient's glucose levels will be monitored carefully with basal insulin levels reduced by 10% when fasting glucose levels fall below 80 mg/dL. If premeal glucose levels are trending downward from baseline a 10% reduction in both the meal in which the premeal glucose level is below 100 mg/dL and the meal prior to that meal. Any symptomatic lows must be immediately reported with the Physician Investigator, to appropriately reduce either the basal or bolus insulin with the goal of glucose levels in the 100 mg/dL range before meals and 140 mg/dL range 2 hours after meals.

In another embodiment, the present invention provides a method for treating new onset or existing type 1 diabetes and LADA through ex vivo administration of new beta cells, islets or stem cells, cadaveric islets or beta cells, or those formed by contacting extra-ductal cells or pluripotent stem cells in culture delivered to a person with type 1 diabetes in combination with a BRM (and optionally in combination with other immune agents) to protect new beta cells from immune attack. In embodiments, the new beta cells are administered to a patient in combination with a BRM and one or more immune tolerance agents to protect the new beta cells delivered to the patient with diabetes from autoimmune destruction. The other beta regeneration agent(s) may include, but are not limited to Reg Peptides, Optimized Reg Peptide formulations and/or agents that bind to the human Reg Receptor. Alternatively or in addition, human cadaveric islet or beta cells may be delivered intravenously or intra-arterially or via an encapsulation method in which a BRM will be used to protect insulin producing cells from immune attack. The BRM may be used with one or more immune tolerance agents which may include, but are not limited to, hOKT3γ1, ChAglyCD3, Rapamycin, Tacrolimus, Etanercept, Alefacept, Belatacept, Diapep277, a tuberculosis vaccine, Glutamic Acid Decarboxylase 65 (GAD65) vaccine; Bacillus Calmette-Guérin Vaccine, Mycophenolate Mofetil alone or in combination with Daclizumab; Rituximab; Campath-1H, lysofylline; antithymocyte globulin, Proleukin and the combination of Proleukin and Rapamune, Vitamin D, IBC-VSO vaccine, Ex vivo Expanded Human Autologous CD4+CD127lo/−CD25+ Polyclonal Regulatory T Cells; a vaccine using CD4⁺CD25⁺ antigen-specific regulatory T cells, Interleukin-1 Receptor Antagonist (anakinra), and/or Alfa-2a 1-Antitrypsin.

In one embodiment, the immune tolerance agent is administered to patients with type 1 diabetes or LADA simultaneously with the administration of new beta cells generated by ex vivo production to protect the new beta cells from autoimmune destruction. In another embodiment, an immune tolerance agent is administered to patients with type 1 diabetes or LADA beginning prior to the time that they are administered the new beta cells generated by ex vivo production to protect the new beta cells from autoimmune destruction.

Embodiments of the invention also include methods of protecting beta cells using a BRM such as oral alfa-2a interferon, which can also be used with pancreatic beta cell generation including both in vivo and ex vivo beta cell generation. Embodiments also include methods for treating a condition that is associated with or is a risk factor for impaired glucose homeostasis. A BRM may also be used to treat or prevent a condition associated with or which is a risk factor for impaired glucose homeostasis which may include, but is not limited to new onset and previously existing type 1 and 2 diabetes in which the patient has characteristics of type 1 diabetes and may test positive for antibodies associated with type 1 diabetes, Latent Autoimmune Diabetes of Adulthood (LADA), those at risk for type 1 diabetes, including but not limited to those who have the diagnosis of diabetes or Pre-diabetes, but with positive autoimmune antibodies markers including Glutamic Acid Decarboxylase-65 antibody and ZNT8.

In one embodiment, patients with a condition that is associated with or is a risk factor for impaired glucose homeostasis are administered beta cells, islets or mesenchymal stem cells generated from ex vivo production in combination with a BRM and/or in combination with another beta regeneration agent or agents. The beta cells may be generated from extra-islet ductal tissue or pluripotent cells contacted in an ex vivo culture and/or in combination with another beta regeneration agent(s) using cell culture techniques known in the art. The other beta regeneration agent(s) may include, but are not limited to Reg Peptide(s) and includes formulations, derivatives, optimized forms and peptidomimetics of Reg Peptides, which may be used in combination with a BRM and/or any immune agent. In another embodiment, patients are administered beta cells generated from ex vivo production or in combination with another beta regeneration agent(s) as well as a BRM in combination with one or more immune tolerance agents to protect new ex vivo-generated beta cells from immune attack. The BRM and immune tolerance agent may be administered to the patient before and/or in parallel (i.e. simultaneously) with the administration of the new beta cells.

In another embodiment, the invention provides a method of treating a condition that is associated with risk for onset of type 1 diabetes, the presence of any autoimmunity factor such as GAD65, insulin autoantibodies, ZNT8 antibodies IA2 Antibodies or abnormal glucose tolerance in a first degree relative of a patient with type 1 diabetes. The BRM may be administered alone or in combination with one or more islet and/or beta cell regeneration or replacement therapies which may include, but are not limited to Reg Peptide(s), including formulations, derivatives, optimized forms and peptidomimetics of the Reg Peptides, small molecules made to the Reg receptor and binding region of the Reg receptor or stimulatory antibodies to the Reg receptor, islet transplants, beta transplants, stem cell transplants or devices that encapsulate beta cells, islets or stem cells. The small molecule or stimulatory antibody may have binding activity against the protein of SEQ ID NO:6 or the peptide of SEQ ID NO:9, or both, or against any portion within these sequences.

The other beta regeneration agent(s) may be administered to the subject in an amount that is effective for generating new beta cells in the pancreas of the subject and/or reducing or preventing symptoms of the condition. The subject may be diabetes drug naïve or on one or more types of insulin.

Embodiments of the compositions and methods of the invention provide the BRM as interferon alfa-2a or pegylated interferon alfa-2a at a dose of 1 IU to 50,000 IU, including 1, 10, 25, 50, 75, 100, 200, 300, 400, 500, 600, 700, 800, 900, 1000, 1250, 1500, 1750, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000 IU, or any range encompassing or including these values such as 4000 to 6000 IU, 3000 to 7000 IU, 2000 to 8000 IU, 2500 to 7500 IU, 1000 to 10000 IU, 2500 to 10000 IU, 1000 to 25000 IU, 1000 to 30000 IU, 1000 to 40000 IU, 1000 to 50000 IU, and so on. In some embodiments, interferon alfa-2a is provided at a dose of about 5000 IU.

BRM embodiments of the invention may be formulated or administered to a patient in pharmaceutically acceptable carriers, such as, for example, oral solutions, oral suspensions, tablets, capsules, ointments, elixirs, and injectable compositions.

Embodiments of compositions of the invention provide a BRM or BRMs in a pharmaceutically acceptable carrier. By “pharmaceutically acceptable” is meant a material that is not biologically or otherwise undesirable, i.e., the material may be administered to a subject, along with the BRM(s), without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered topically, orally, or parenterally. For example, the compositions can be administered extracorporeally, intracranially, intravaginally, intraanally, subcutaneously, intradermally, intracardiac, intragastric, intravenously, intramuscularly, by intraperitoneal injection, transdermally, intranasally, or by inhalation. As used herein, “intracranial administration” means the direct delivery of substances to the brain including, for example, intrathecal, intracisternal, intraventricular or trans-sphenoidal delivery via catheter or needle.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

As used herein, “topical intranasal administration” means delivery of the compositions into the nose and nasal passages through one or both of the nares and can comprise delivery by a spraying mechanism or droplet mechanism, or through aerosolization of the BRM(s). Administration of the compositions by inhalant can be through the nose or mouth via delivery by a spraying or droplet mechanism. Delivery can also be directly to any area of the respiratory system (e.g., lungs) via intubation.

The exact amount of the compositions required will vary from subject to subject, depending on the species, age, weight and general condition of the subject, the severity of the condition being treated, the BRM used, its mode of administration and the like. Thus, it is not possible to specify an exact amount for every composition. However, an appropriate amount can be determined by one of ordinary skill in the art using only routine experimentation given the teachings herein.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8, from about 7 to about 7.5. Further carriers include sustained release preparations such as semipermeable matrices of solid hydrophobic polymers containing the BRM, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, gels (e.g., poloxamer gel), drops, controlled-release compositions, timed release compositions, suppositories, sprays, liquids and powders. Conventional pharmaceutical carriers, aqueous, powder or oily bases, thickeners and the like may be necessary or desirable. The disclosed compositions can be administered, for example, in a microfiber, polymer (e.g., collagen), glasses, nanosphere, aerosol, lotion, cream, fabric, plastic, tissue engineered scaffold, matrix material, tablet, implanted container, powder, oil, resin, wound dressing, bead, microbead, slow-release compounds, timed-release compounds, capsule, injectables, intravenous drips, pump device, silicone implants, or any bio-engineered materials.

Compositions for oral administration include powders or granules, suspensions or solutions in water or non-aqueous media, capsules, sachets, or tablets. Thickeners, flavorings, diluents, emulsifiers, dispersing aids or binders may be desirable. Pharmaceutically acceptable carriers include fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are flow-regulating agents and lubricants, for example, silica, talc, stearic acid or salts thereof, such as magnesium stearate or calcium stearate, and/or polyethylene glycol. In one embodiment, dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Slow dissolving polymers such as poly(bis(p-carboxyphenoxy)-propane:sebacic acid—CCP:SA) may also be used to generate wafers or beads that control or time the release of the composition. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active ingredients in the form of granules or nanoparticles which may optionally be mixed with fillers such as lactose, binders such as starches, and/or lubricants such as talc or magnesium stearate and, optionally, stabilizers. In one embodiment, the BRM(s) of this disclosure are dissolved or suspended in suitable liquids, such as fatty oils, or liquid paraffin, optionally with stabilizers.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Fatty oils may comprise mono-, di- or triglycerides. Mono-, di- and triglycerides include those that are derived from C6, C8, C10, C12, C14, C16, C18, C20 and C22 acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof. Exemplary triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

Pharmaceutical compositions comprising triglycerides may further comprise lipophilic and/or hydrophilic surfactants which may form clear solutions upon dissolution with an aqueous solvent. One such surfactant is tocopheryl polyethylene glycol 1000 succinate (vitamin E TPGS). Examples of such compositions are described in U.S. Pat. No. 6,267,985.

Suitable formulations for parenteral administration include aqueous solutions of the BRM(s) in water soluble form, for example, water-soluble salts and alkaline solutions. In addition, suspensions of the BRM(s) as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions may be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams may be formulated from a mixture of mineral oil, self-emulsifying beeswax and water in which the BRM(s), dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a suspension of the BRM(s) in a vegetable oil such as almond oil with warm soft paraffin and allowing the mixture to cool. A typical example of such an ointment is one which includes about 30% almond oil and about 70% white soft paraffin by weight.

Lotions may be conveniently prepared by preparing a suspension of the BRM(s) in a suitable high molecular weight alcohol such as propylene glycol or polyethylene glycol.

Suitable routes of administering the pharmaceutical preparations include oral, rectal, topical (including dermal, buccal and sublingual), vaginal, parenteral (including subcutaneous, intramuscular, intravenous, intradermal, intrathecal, intratumoral, and epidural) and by naso-gastric tube. It will be understood by those skilled in the art that the preferred route of administration will depend upon the condition being treated and may vary with factors such as the condition of the recipient.

Possible pharmaceutical preparations which can be used rectally include, for example, suppositories, which consist of a combination of the BRM(s) this disclosure with a suppository base. Suitable suppository bases are, for example, natural or synthetic triglycerides, or paraffin hydrocarbons. In addition, it is also possible to use gelatin rectal capsules which consist of the BRM(s) of this disclosure with a base. Possible base materials include, for example, liquid triglycerides, polyethylene glycols, or paraffin hydrocarbons.

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, immunosuppression, and the like. Generally, the dosage will vary with the age, condition, sex and extent of the disease in the patient, route of administration, or whether other drugs are included in the regimen, and can be determined by one of skill in the art. The dosage can be adjusted by the individual doctor in the event of any counter indications. Dosage can vary, and can be administered in one or more dose administrations daily, for one or several days. Guidance can be found in the literature for appropriate dosages for given classes of pharmaceutical products. The range of dosage largely depends on the application of the compositions herein, severity of condition, and its route of administration.

Any embodiment of the composition may formulate the BRM(s) with a beta cell regeneration agent, such as a Reg peptide. In other embodiments, the beta cell regeneration agent is formulated separately. The beta cell regeneration agent may be a Reg peptide or optimized Reg peptide. As used herein, an “optimized” Reg peptide is a Reg peptide that is modified to increase the efficacy of the peptide when administered to a subject through desirable pharmacokinetic, pharmaceutical, or immunological properties such as to increase stability of the peptide in a biological fluid such as plasma, increase its solubility (such as in an aqueous medium), increase its protease resistance, reduce its immunogenicity, increase Tmax (known in the pharmaceutical arts as the time taken to reach Cmax, which is the maximum concentration in plasma after administration to a subject), and/or increase its bioavailability. Optimization of the peptide may include optimization for any other pharmacokinetic or pharmaceutical parameter which is desirable for optimal efficacy in the subject administered the peptide. Such modifications may include any combination of blocking with an n-terminal acetyl group and/or a c-terminal amide group, cyclization, dimerization, providing an additional n-terminal cysteine residue, pegylation, and the like. Examples of Reg peptides and optimized Reg peptides are described in previous patents issued to the present inventor such as U.S. Pat. Nos. 8,911,776 and 9,321,812, each of which is incorporated by reference in its entirety. Embodiments of Reg peptides are shown in SEQ ID NOS:1, 4, 7, 8, 11, 12, and 14 of this disclosure. Embodiments of optimized Reg peptides are shown in SEQ ID NOS:15-39 of this disclosure. The optimized peptides shown in SEQ ID NOS:15-39 are included as examples of the types of modifications that may increase stability, increase solubility, increase protease resistance, reduce immunogenicity, increase Tmax, and/or increase bioavailability, and should not be construed as limiting. A skilled artisan can appreciate variations of the optimized peptides shown in SEQ ID NOS:15-39 which fall within the scope of the invention.

The Reg peptides may be produced through recombinant molecular biology techniques or solid phase synthesis techniques. Recombinant molecular biology techniques include those described in Molecular Cloning: A Laboratory Manual, Green and Sanbrook, 2012. Solid-phase synthesis techniques are described in Merrifield, in J. Am. Chem. Soc., 15:2149-2154 (1963), M. Bodanszky et al., (1976) Peptide Synthesis, John Wiley & Sons, 2d Ed.; Kent and Clark-Lewis in Synthetic Peptides in Biology and Medicine, p. 295-358, eds. Alitalo, K., et al. Science Publishers, (Amsterdam, 1985); as well as other reference works known to those skilled in the art such. A summary of peptide synthesis techniques may be found in J. Stuart and J. D. Young, Solid Phase Peptide Synthelia, Pierce Chemical Company, Rockford, Ill. (1984), which is incorporated herein by reference. The synthesis of peptides by solution methods may also be used, as described in The Proteins, Vol. II, 3d Ed., p. 105-237, Neurath, H. et al., Eds., Academic Press, New York, N.Y. (1976). Appropriate protective groups for use in such syntheses will be found in the above texts, as well as in J. F. W. McOmie, Protective Groups in Organic Chemistry, Plenum Press, New York, N.Y. (1973), which is incorporated herein by reference. In general, these synthetic methods involve the sequential addition of one or more amino acid residues or protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine. Block synthesis techniques may also be applied to both the solid phase and solution methods of peptide synthesis. Rather than sequential addition of single amino acid residues, preformed blocks comprising two or more amino acid residues in sequence are used as either starting subunits or subsequently added units rather than single amino acid residues. Alternative or additional peptide synthesis methods and techniques can be found in Peptide Chemistry: A Practical Textbook: 2nd Edition, Miklos Bodanszky, 1993.

Reg peptides may also be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (see, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting optimized is accomplished using conventional procedures, such as preparative HPLC using gel permeation, partition and/or ion exchange chromatography. The choice of appropriate matrices and buffers are well known in the art and so are not described in detail herein.

Protocols for blocking peptides with acetyl and amide groups are known in the art and can be found in a number of protein protocol textbooks known in the art. Specific examples include those published in Methods in Molecular Biology, Vol. 35: Peptide Synthesis Protocols, Chapter 8: Site-Specific Chemical Modification Procedures, Edited by M W Pennington and B M Dunn, 1994, as well as U.S. Pat. Nos. 4,708,934, 5,503,989, U.S Patent Application Publication No. US 20060127995. Alternative or additional blocking procedures can be found in Peptide Chemistry: A Practical Textbook: 2nd Edition, Miklos Bodanszky, 1993.

Inert polymer molecules such as high molecular weight polyethylene glycol (PEG) can be attached to a Reg peptide of this disclosure or an analog or derivative thereof with or without a multifunctional linker either through site-specific conjugation of the PEG to the N- or C-terminus of the protein or via epsilon-amino groups present on lysine residues. Linear or branched polymer derivatization that results in minimal loss of biological activity can be used. The degree of conjugation can be closely monitored by SDS-PAGE and mass spectrometry to ensure proper conjugation of PEG molecules. Unreacted PEG can be separated from peptide-PEG conjugates by size-exclusion or by ion-exchange chromatography.

The optimized peptides may also be PEGylated at cysteine residues through maleimide chemistry. Maleimide-activated PEG reacts with the thiols of cysteine residues of protein and to form stable thioether linkages and are highly stable against hydrolysis. The maleimide moiety reacts rapidly with the thiol group without hydrolysis around neutral pH. Protocols for creating maleimide-activated PEG constructs may be found in Schumacher et al., In Situ Maleimide Bridging of Disulfides and a New Approach to Protein PEGylation, Bioconjugate Chem., 2011, 22 (2), pp 132-136, Doherty et al., Site-Specific PEGylation of Engineered Cysteine Analogs of Recombinant Human Granulocyte-Macrophage Colony-Stimulating Factor, Bioconjug Chem. 2005; 16(5): 1291-1298, US Patent Application Publication No. 20090298746 Al, European Patent No. EP 1881850 B1, European Patent No. EP 2178900 B1.

Embodiments of the invention also include pharmaceutical compositions, which formulate a BRM in multiple formulations to derive the greatest stability for oral delivery. In one embodiment, the invention uses 1.2 mg of 25% human albumin in a quantity of 1.2 mg/mL in an actual volume of 4.32 mL with 6000 IU/mL of interferon Alfa-2a in a volume of 0.45 mL with 900 mL of 0.9% Sodium Chloride.

An exemplary embodiment of the present invention comprises an oral BRM formulation such as 5000 IU of oral interferon or a PEGylated version of alfa-2a interferon given orally as a protector of beta cells, islets, mesenchymal stem cells formed or administered to a patient with type 1 diabetes, pre type 1 diabetes or at risk for developing diabetes as indicated by immune markers including but not limited to GAD65, insulin autoantibodies, ZNT8 antibodies IA2 Antibodies. Alternatively or in addition, the oral BRM formulation may be given to patients with immune markers who do not have glucose intolerance.

In another embodiment, the invention provides a combination product comprising a BRM such as oral interferon alfa-2a or oral PEGylated interferon alfa-2a combined with or more one immune agents to protect beta cells in a patient with type 1 diabetes or pre type 1 diabetes or a patient with autoimmunity at risk for diabetes. The BRM may be used with any islet neogenesis agent in combination with another beta regeneration agent or agents inclusive but not limited to Reg Peptides, Optimized Reg Peptide formulations and/or agents that bind to the human Reg Receptor. The BRM may be used in combination with cadaveric islet transplants, ex vivo made islets, beta cells or mesenchymal stem cells or devices housing beta cells, islets, stem cells for use in patients with type 1 diabetes.

Pancreatic islet transplantation is known in the art. Pancreatic islet allo-transplantation is a procedure which utilizes islets from a deceased organ donor. Briefly, the islets are removed from the donor by injecting a collagenase solution into the pancreatic duct. The pancreas is cut into small pieces and transferred to a Ricordi Chamber, which breaks down the pancreas further through a combination of mechanical forces and enzymatic digestion. The liberated islets are then removed from the solution and subject to purification in which exocrine tissue is removed. During transplantation into the recipient, typically a radiologist guides a catheter into the portal vein under assistance by ultrasound and radiography. The purified islets are then infused into the liver via the catheter. Transplant patients typically receive two infusions with an average of 400,000 to 500,000 islets per infusion (see https://www.niddk.nih.gov/health-information/diabetes/overview/insulin-medicines-treatments/pancreatic-islet-transplantation). Once implanted, the beta cells in these islets begin to make and release insulin. However, traditionally, the need for immunosuppressive medications and a shortage of donors has limited this procedure as a large-scale treatment for type I diabetes.

Encapsulation of islets is a strategy known in the art to address the need to protect the transplanted islets from the immune system and involves coating or surrounding the islet cells or tissue in a semipermeable biocompatible material that permits the passage of nutrients, oxygen, and hormones while blocking cells and substances of the immune system from recognizing and destroying the transplant. Encapsulation technologies and devices are reviewed in the scientific literature (see Tejal Desai and Lonnie D. Shea, Nature Reviews Drug Discovery 16, 338-350 (2017); and Rahul Krishnan, et al., Rev Diabet Stud. 2014 Spring; 11(1): 84-101; each incorporated herein by reference), and have been described in the patent literature as well (for example, see U.S. Pat. Nos. 5,869,077; 7,427,415; and 6,287,558).

The islets or beta cells employed in compositions and methods of the invention may include allogenic, autogenic, xenogeneic islets or beta cells, or any genetically modified variant of allogenic, autogenic, xenogeneic islets or beta cells. For example, in some embodiments, a vector is introduced into the islets or beta cells which vector includes one or more genes under the control of a constitutive or inducible promoter. The one or more genes may augment or enhance the islets or beta cells in terms of viability, insulin secretion, immunocompatibility, and the like. Such gene therapy vectors are known in the art and need not be reviewed here.

The stem cells employed in compositions and methods of the invention may include embryonic stem cells, adult stem cells, induced pluripotent stem cells, human adult bone-marrow derived cells, mesenchymal stem cells, human amniotic membrane-derived mesenchymal cells, mammalian stem cells, and ectodermal stem cells, umbilical cord stem cells, or other stem cells and may include resident populations of endogenous stem cells that exist within the adult pancreas. In embodiments, the stem cells may be transformed into beta cells ex vivo through contact with one or more Reg peptides or optimized Reg peptides or may be administered in vivo for transformation into beta cells. The stem cells or beta cells may be may be administered intravenously, subcutaneously, intra-arterial and delivery including delivery to the pancreas, liver or other appropriate targets to optimize efficacy.

In one embodiment, the kits of the present invention include one or more compositions of the present invention together with information which informs a user of the kit, by words, pictures, and/or the like, that use of the kit will treat various conditions associated with or a risk factor for impaired glucose homeostasis (e.g. type 1 in patient with a family history of type 1 diabetes who expresses impaired glucose tolerance and/or the expression of autoimmune antibodies indicating a high risk for the development of type 1 diabetes, which include ZNT8 antibodies, GAD-65 antibodies, IA-2 antibodies, insulin antibodies or others). The use of a BRM may be used alone or in combination with a therapeutically effective dose of another beta regeneration agent(s) including but not limited to Reg Peptides, Optimized Reg Peptides or Reg Peptide peptidomimetics and other Reg formulations or with islet transplants, beta transplants, stem cell transplants or devices housing islets, beta cells or stem cells. A BRM may also be used in a therapeutically effective dosage alone or with immune tolerance agent(s).

Further embodiments provide a kit for measuring endogenous insulin, insulin-requirements, antibodies to Islet-cell autoantibodies 512 (ICA512)/islet antigen-2 (IA-2), Glutamic acid decarboxylase (GAD) autoantibodies, ZNT8 Antibodies, Insulin autoantibodies (ICA512/IA-2) at baseline and during and after treatment.

### EXAMPLES

### Example 1

A patient with type 1 diabetes is administered a daily subcutaneous 300 mg injection of an optimized form of a 14 or 15 amino acid Reg peptide along with 5000 IU of oral interferon in saline and/or with human albumin or in another formulation for oral usage. The Reg peptide may be administered subcutaneously on a daily basis, in an oral preparation that will initially be given on a daily basis, as a longer acting subcutaneous therapy, or delivered via an encapsulated device that slowly releases daily dosages of the peptide, in combination with 5000 IU of oral interferon. Based on blood glucose levels, exogenous insulin injection dosages are tapered over a period of 12 weeks based on glucose levels to a point that insulin is no longer required. The patient will then be continued on oral interferon 5000 IU per day with Reg peptide reduced to weekly injections or oral delivery or reduced dosage by an encapsulated delivery system.

### Example 2

A patient with type 1 diabetes is given a cadaveric islet transplantation via the umbilical vein and given 5000 IU oral interferon alfa 2-a in saline and/or with human albumin along with any other immunotherapy. Based on blood glucose levels, insulin is tapered over a period of 12 weeks based on glucose levels to a point that insulin is no longer required. The patient will be treated with oral interferon alfa-2a at a dosage of 5000 IU per day and may also be treated daily followed by weekly subcutaneous injections of a Reg peptide until the patient is insulin independent. Oral interferon with cadaveric islet transplant may also be used without the administration of Reg peptide.

### Example 3

A patient with type 1 diabetes receives an implanted device containing islets, beta or stem cells, and is given 5000 IU oral interferon alfa-2a in saline and/or with human albumin along with any other immunotherapy. Based on blood glucose levels, insulin is tapered off over a period of 12 weeks based on glucose levels to a point that insulin is no longer required. The patient will then be continued on oral interferon 5000 IU per day and may also be simultaneously treated daily followed by weekly subcutaneous injections of a Reg peptide until the patient is insulin independent at which time the dosing of Reg peptide may be reduced, but with continuation of oral interferon to prevent autoimmune attack on beta cells formed from Reg peptide, or from implanted beta cells, islets or stem cells.

### Example 4

A patient who develops glucose intolerance who has any autoimmune markers which place the patient at risk for type 1 diabetes including GAD65, insulin autoantibodies, ZNT8 antibodies IA2 Antibodies or has a family history of a first degree relative with type 1 diabetes is given a daily dosage of 5000 IU of oral interferon 2a for prevention of type 1 diabetes.

### Example 5

A patient who does not have a history of glucose intolerance, but has GAD65, insulin autoantibodies, ZNT8 antibodies IA2 Antibodies present or a family history of type 1 diabetes is given 5000 IU per day of oral alfa-2a interferon to prevent the onset of type 1 diabetes.

### Example 6

A patient presenting with initial symptoms of conditions that may have an autoimmune basis for which there is no successful treatment or available treatment has not stopped the progression of the disease and may include, Multiple Sclerosis, ALS, forms of Parkinson's, immune system disorders that attack the basal ganglia including pediatric autoimmune neurobiological disorders, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes, anti-myelin associated glycoprotein (MAG)-related neuropathies, Combined Sensorimotor Neuropathy in Rheumatoid Arthritis, Juvenile Rheumatoid Arthritis is given 5000 IU per day of oral interferon alfa-2a.

### OTHER EXAMPLES

Once patients requiring insulin are begun on an oral BRM in combination with an islet neogenesis agent, beta regeneration agent, beta, islet or stem cell transplant, or device housing islets, beta cells or stem cells, glucose must be levels will be monitored carefully and the basal and bolus level of insulin modified. Islet neogenesis can only optimally occur when the patient is mildly hyperglycemia, thus basal and bolus levels of insulin will need to be producing and endogenous insulin is made. Basal (pump delivered insulin or injectable insulin). Exogenous levels of insulin should be reduced by 10% when fasting glucose levels fall below 100 mg/dL for a given meal. If premeal glucose levels continue to trend downward from baseline and are lower than 100 mg/dL, a 20% reduction in exogenous insulin injections will continue for that that specific meal. If the premeal insulin is than 70 mg/mL, the exogenous insulin level will be reduced by 20% for those particular meals. For fasting glucose below the level of 125 mg/dL, the basal dosage of basal insulin will be reduced by 10% and be continued at that lower dosage. If the fasting glucose continues to fall below 100 mg/dL, the basal dosage will continued to be dropped by 10% on the subsequent basal dosages. Each time there is a level of less than 100 mg/dL prior to a meal, the premeal insulin, drop by 10% and if lower than 70 mg/dL, the dosage will be decreased by 20%. Any symptomatic episodes of hypoglycemia with glucose less than 55 mg/ml, the patient should follow hypoglycemia protocol and must be immediately reported to the physician, to lower either the basal or bolus insulin or both with the goal of maintaining glucose levels in the 100 mg/dL range before meals and 140 mg/dL range 2 hours after meals.

Embodiments also provide methods and pharmaceutical compositions for insulin independence from exogenous insulin in a patient with type 1 patient using a BRM with a beta cell agonist including, but not limited to Reg peptides, derivatives, formulations and peptidomimetics to the Reg receptor, islet transplants, beta or mesenchymal stem cell transplants, or an implantable device housing islets, stem cells or beta cells. Once patients are begun on a BRM with a beta or islet regeneration agent, islet transplant, beta or mesenchymal stem cell transplant, etc., glucose levels must be monitored carefully. Once patients requiring insulin are begun on an oral BRM in combination with an islet neogenesis agent, beta regeneration agent, beta, islet or stem cell transplant, or device housing islets, beta cells or stem cells, glucose must be levels will be monitored carefully and the basal and bolus level of insulin modified. Islet neogenesis can only optimally occur when the patient is mildly hyperglycemia, thus basal and bolus levels of insulin will need to be producing and endogenous insulin is made. Basal (pump delivered insulin or injectable insulin). Exogenous levels of insulin should be reduced by 10% when fasting glucose levels fall below 100 mg/dL for a given meal. If premeal glucose levels are continuing to trending downward from baseline and are lower than 100 mg/dL, a 20% reduction in exogenous insulin injections will continue for that that specific meal. If the premeal insulin is than 70 mg/mL, the exogenous insulin level will be reduced by 20% for those particular meals. For fasting glucose below the level of 125 mg/dL, the basal dosage of basal insulin will be reduced by 10% and be continued at that lower dosage. If the fasting glucose continues to fall below 100 mg/dL, the basal dosage will continued to be dropped by 10% on the subsequent basal dosages. Each time there is a level of less than 100 mg/dL prior to a meal, the premeal insulin, drop by 10% and if lower than 70 mg/dL, the dosage will be decreased by 20%. Any symptomatic episodes of hypoglycemia with glucose less than 55 mg/ml, the patient should follow hypoglycemia protocol and must be immediately reported to the physician, to lower either the basal or bolus insulin or both with the goal of maintaining glucose levels in the 100 mg/dL range before meals and 140 mg/dL range 2 hours after meals.

Once patients are begun on a BRM with beta regeneration agent, islet transplant, beta or mesenchymal stem cell transplant or with a device housing islets, stem cells or beta cells, glucose levels will be monitored carefully with basal and bolus insulin levels adjusted. Islet neogenesis can only optimally occur when the patient is mildly hyperglycemia, thus basal and bolus levels of insulin will need to be producing and endogenous insulin is made. Basal (pump delivered insulin or injectable insulin). Exogenous levels of insulin should be reduced by 10% when fasting glucose levels fall below 100 mg/dL for a given meal. If premeal glucose levels are continuing to trending downward from baseline and are lower than 100 mg/dL, a 20% reduction in exogenous insulin injections will continue for that that specific meal. If the premeal insulin is than 70 mg/mL, the exogenous insulin level will be reduced by 20% for those particular meals. For fasting glucose below the level of 125 mg/dL, the basal dosage of basal insulin will be reduced by 10% and be continued at that lower dosage. If the fasting glucose continues to fall below 100 mg/dL, the basal dosage will continued to be dropped by 10% on the subsequent basal dosages. Each time there is a level of less than 100 mg/dL prior to a meal, the premeal insulin, drop by 10% and if lower than 70 mg/dL, the dosage will be decreased by 20%. Any symptomatic episodes of hypoglycemia with glucose less than 55 mg/ml, the patient should follow hypoglycemia protocol and must be immediately reported to the physician, to lower either the basal or bolus insulin or both with the goal of maintaining glucose levels in the 100 mg/dL range before meals and 140 mg/dL range 2 hours after meals.

Islet neogenesis can only optimally occur when the patient is mildly hyperglycemia, thus basal and bolus levels of insulin will need to be producing and endogenous insulin is made. Basal (pump delivered insulin or injectable insulin). Exogenous levels of insulin should be reduced by 10% when fasting glucose levels fall below 100 mg/dL for a given meal. If premeal glucose levels are continuing to trending downward from baseline and are lower than 100 mg/dL, a 20% reduction in exogenous insulin injections will continue for that that specific meal. If the premeal insulin is than 70 mg/mL, the exogenous insulin level will be reduced by 20% for those particular meals. For fasting glucose below the level of 125 mg/dL, the basal dosage of basal insulin will be reduced by 10% and be continued at that lower dosage. If the fasting glucose continues to fall below 100 mg/dL, the basal dosage will continued to be dropped by 10% on the subsequent basal dosages. Each time there is a level of less than 100 mg/dL prior to a meal, the premeal insulin, drop by 10% and if lower than 70 mg/dL, the dosage will be decreased by 20%.

Any symptomatic episodes of hypoglycemia with glucose less than 55 mg/ml, the patient should follow hypoglycemia protocol and must be immediately reported to the physician, to lower either the basal or bolus insulin or both with the goal of maintaining glucose levels in the 100 mg/dL range before meals and 140 mg/dL range 2 hours after meals.

Any symptomatic lows must be immediately reported to the physician, to appropriately lower either the basal or bolus insulin with the goal of glucose levels in the 100 mg/dL range before meals and 140 mg/dL range 2 hours after meals and also be tapered as glucose levels and hemoglobin A1C fall into the normal range resulting from this invention.

Embodiments also include methods and pharmacologic compositions for improved glycemic control and ability to restore normoglycemic among diabetes drug naïve patients such as a newly diagnosed or previously diagnosed type 1 diabetes patient who is currently on no pharmaceutical treatment for diabetes, in which a BRM can be started with a beta or islet regeneration agent, islet transplant, beta or mesenchymal stem cell transplant or with a device housing islets, stem cells or beta cells along with the usage of insulin. The glucose goals would be in the 100 mg/dL range before meals and 140 mg/dL two hours after meals.

Because there are numerous redundant mechanisms to prevent hypoglycemia, which do not allow for new beta cell formation under normal physiological conditions and even as glucose levels approach normal levels, there is limited if any ability to generate new islets, thus risk for hypoglycemia must be prevented and exogenous insulin withdrawn as glucose levels approach normal and Hemoglobin A1C normalizes.

### ADDITIONAL EMBODIMENTS

### Embodiment 1a

A method of treating a condition that is associated with or is a risk factor for type 1 diabetes, new onset type 1 or existing type 1 diabetes, or latent autoimmune diabetes of adulthood (LADA), or any form of diabetes, Pre-diabetes or those at risk for diabetes who exhibit physical signs of type 1 diabetes or autoimmune antibodies associated with diabetes including, but not limited to Islet-cell autoantibodies 512 (ICA512)/islet antigen-2 (IA-2), Glutamic acid decarboxylase (GAD) autoantibodies, ZNT8 Antibodies, Insulin autoantibodies (ICA512/IA-2) and treated by administering a BRM such as oral interferon alfa-2a in the amount of 5000 IU per day and may be used with an islet or beta cell regeneration agent, islet, beta or stem cell transplant or a device housing islets, beta cells or stem cells into a patient with type 1 diabetes, LADA, pre-type 1 diabetes or a patient without glucose abnormalities, but with a family history of type 1 diabetes or the presence of Islet-cell autoantibodies 512 (ICA512)/islet antigen-2 (IA-2), Glutamic acid decarboxylase (GAD) autoantibodies, ZNT8 Antibodies, Insulin autoantibodies (ICA512/IA-2) with a given amount of an islet neogenesis agent such as a 14 or 15 amino acid Reg peptide delivered in a subcutaneous dosage of 60-300 mg daily to the subject that is effective for generating new islets.

### Embodiment 1b

The method of any preceding embodiment, further comprising the step of administering an amount of a BRM to the subject that is effective for protecting the new beta cells from destruction from the immune system and/or reducing or preventing symptoms of the condition.

### Embodiment 1c

The method of any preceding embodiment, further comprising the step of administering an amount a BRM alone or with at least one beta regeneration agent or islet neogenesis agent or islet, beta cell or stem cell transplant or with a device that houses islets, stem cells or beta cells that is effective for generating and maintaining new beta cells in the pancreas of the subject and/or reducing or preventing symptoms of the condition.

### Embodiment 1d

The method of any preceding embodiment, further comprising the step of administering an amount of a BRM alone or with at least one beta regeneration agent or islet neogenesis agent or islet, beta cell or stem cell transplant or with a device that houses islets, stem cells or beta cells that is effective for generating and maintaining new beta cells in the pancreas that is effective for generating new beta cells in the pancreas of the subject and/or reducing or preventing symptoms of the condition.

### Embodiment 2a

The method of any preceding embodiment, wherein the BRM is alfa-2a interferon 2a or PEGylated alfa-2a.

### Embodiment 2b

The method of any preceding embodiment, wherein a BRM is used alone or with at least one beta regeneration agent or islet neogenesis agent or islet, beta cell or stem cell transplant or with a device that houses islets, stem cells or beta cells that is effective for generating and maintaining new beta cells in the pancreas of the subject and/or reducing or preventing symptoms of the condition and one or more other immune tolerance agent(s) are used and may include but are not limited to Cyclosporine, hOKT3γ1, ChAglyCD3, Rapamycin, Tacrolimus, Etanercept, Alefacept, Belatacept, Diapep277, a tuberculosis vaccine, Glutamic Acid Decarboxylase 65 (GAD65) vaccine; Bacillus Calmette-Guérin Vaccine, Mycophenolate Mofetil alone or in combination with Daclizumab; Rituximab; Campath-1H, lysofylline; antithymocyte globulin, Proleukin and the combination of Proleukin and Rapamune, Vitamin D, IBC-VSO vaccine, Ex vivo Expanded Human Autologous CD4+CD127lo/−CD25+ Polyclonal Regulatory T Cells; interferon-alfa-2a; a vaccine using CD4⁺CD25⁺ antigen-specific regulatory T cells, Interleukin-1 Receptor Antagonist (anakinra), and Alfa-2a 1-Antitrypsin.

### Embodiment 2d

The method of any preceding embodiment, wherein a BRM is used with a beta regeneration or islet neogenesis agent such as a Reg Peptide, or a formulation, derivative, optimized form or peptidomimetic of a Reg Peptide.

### Embodiment 2e

The method of any preceding embodiment wherein the BRM is an oral preparation, capsule, pill, suspension, or solution.

### Embodiment 3a

The method of any preceding embodiment wherein treatment with a BRM results in reduction in diabetes medication requirements.

### Embodiment 3b

The method of any preceding embodiment, wherein the diabetes medication is insulin.

### Embodiment 4a

The method of any preceding embodiment, wherein the treatment results in insulin independence.

### Embodiment 4b

The method of any preceding embodiment, wherein the condition that is associated with impaired glucose homeostasis is type 1 diabetes or pre-Type 1 diabetes and result in diminution of insulin dosage or insulin independence.

### Embodiment 4c

The method of any preceding embodiment, wherein the subject is diabetes-drug naïve but expresses antibodies indicative of the onset of diabetes and the patient may have pre-type 1 diabetes, abnormalities in glucose metabolism, but may not yet meet the criteria for diabetes.

### Embodiment 4d

The method of any preceding embodiment, wherein the subject has been exposed to diabetes drugs and the patient is later considered to have autoimmune or type 1 diabetes based on clinical signs or autoimmune antibodies and glucose homeostasis is not restored on the current diabetes medication that the patient has been placed and use of methods and therapies within these embodiments is effective in improving glucose metabolism.

### Embodiment 4e

The method of any preceding embodiment, wherein treatment results in reduction in diabetes medication requirements that patient received prior to the treatment described herewithin.

### Embodiment 4f

The method of any preceding embodiment, wherein the diabetes medication is insulin.

### Embodiment 4g

The method of any preceding embodiment, wherein the treatment results in insulin independence.

### Embodiment 5a

The method of any preceding embodiment, wherein the BRM is administered alone or in combination with other therapies.

### Embodiment 5b

The method of any preceding embodiment, wherein the condition that is associated with impaired glucose homeostasis is type 1 diabetes.

### Embodiment 5c

A method for treating a pathology associated specifically with impaired pancreatic function in a subject comprising the step of:

### Embodiment 5c.i

Administering a therapeutically effective amount of a BRM to the subject who has type 1 diabetes, is at risk for type 1 diabetes or has glucose intolerance with autoimmune antibodies associated with type 1 diabetes or the patient has a first-degree relative with diabetes.

### Embodiment 5c.ii

The method of any preceding embodiment, further comprising one or more steps of:

### Embodiment 5c.iii

Intensifying glycemic control in a patient with type 1 diabetes or pre type 1 diabetes;

### Embodiment 5c.iv

Administering oral vitamin D to maintain 25-hydroxyvitamin levels above 40 mg/ml;

### Embodiment 5c.v

Reducing, or tapering off of other diabetes therapies as new beta cell populations are restored; and

### Embodiment 5c.vi

Lowering the dosage of dosages of other diabetes medication, including insulin, is tapered off.

### Embodiment 6a

The method of any preceding embodiment, wherein the pathology associated specifically with impaired pancreatic function is type 1 diabetes.

### Embodiment 6b

The method of any preceding embodiment, where in the pathology is a disease state that has been associated to occur with type 1 diabetes, or found in the family of a patient with type 1 diabetes and may include, but not be limited to conditions for which treatment is ineffective or there is no treatment and the disease is progressive and can lead to death and the usage of oral interferon alfa-2a may halt or slow the progressive nature of the condition and include but are not limited to amyotrophic lateral sclerosis and multiple sclerosis and may be used for other autoimmune disorders that have no treatment including amyotrophic lateral sclerosis (ALS), forms of Parkinson's, immune system disorders that attack the basal ganglia including pediatric autoimmune neurobiological disorders, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis, Juvenile Rheumatoid Arthritis.

### Embodiment 7a

A method for the protecting new beta cells generated from extra-islet ductal tissue or pluripotent stem cells, comprising the steps of:

### Embodiment 7b

In-vivo generation of new islets or beta cells from an islet neogenesis agent, beta regeneration agent or by protection of transplanted islets or beta cells from cadaveric transplants or generated by culturing the extra-islet ductal tissue or pluripotent stem cells ex vivo; and

### Embodiment 7c

Using a BRM to protect beta cells from autoimmune attack whether beta cells are derived from contacting said extra-islet ductal tissue or pluripotent stem cells with a proton pump inhibitor, wherein the amount of proton pump inhibitor is effective for forming beta cells from said extra-islet ductal tissue or pluripotent stem cells.

### Embodiment 7d

The method of embodiment 17a, further comprising the step of contacting said extra-islet ductal tissue or pluripotent stem cells with at least one other beta regeneration agent.

### Embodiment 7e

The method of embodiment 17a, wherein the pluripotent stem cells are embryonic cells, adult somatic stem cells, human adult bone-marrow derived stem cells, umbilical cord stems cells, mesenchymal stem cells, human amniotic membrane-derived mesenchymal cells, mammalian stem cells, ectodermal stem cells, or endogenous stem cells that exist within the adult pancreas.

### Embodiment 8a

The method of any preceding embodiment, wherein the other beta regeneration agent is a Reg Peptide, or a formulation, derivative, optimized form or peptidomimetic of a Reg Peptide.

### Embodiment 8b

A method of treating a condition that is associated with or is a risk factor for impaired glucose homeostasis in a subject selected from new onset type 1 and 2 diabetes, in which antibodies are found or the patient is not responding to type 2 diabetes therapies and requires insulin and may include previously existing type 1 and those categorized as 2 diabetes, who do not respond to therapy and require insulin, latent autoimmune diabetes of adulthood (LADA), those with a family history of type 1 or insulin-requiring diabetes with positive antibodies, the method comprising:

### Embodiment 8c

Culturing the extra-islet ductal tissue or pluripotent stem cells ex vivo;

### Embodiment 8d

Contacting said extra-islet ductal tissue or pluripotent stem cells with a beta regeneration agent in an amount which is effective for forming beta cells from said extra-islet ductal tissue or pluripotent stem cells; and

### Embodiment 8e

Administering the beta cells to the subject.

### Embodiment 9a

The method of embodiment 18a, further comprising the step of contacting said extra-islet ductal tissue or pluripotent stem cells with another beta regeneration agent, which can be the same or a different beta regeneration agent, wherein the amount of other beta regeneration agent is effective for forming beta cells with usage of oral interferon alfa-2a to protect beta cells from autoimmune destruction.

### Embodiment 10a

The method of any preceding embodiment, wherein the pluripotent stem cells are embryonic cells, adult somatic stem cells, human adult bone-marrow derived stem cells, umbilical cord stems cells, mesenchymal stem cells, human amniotic membrane-derived mesenchymal cells, mammalian stem cells, ectodermal stem cells, or endogenous stem cells that exist within the adult pancreas with usage of oral interferon alfa-2a to protect beta cells from autoimmune destruction.

### Embodiment 11a

The method of any preceding embodiment, wherein the other beta regeneration agent is a Reg Peptide, or a formulation, derivative, optimized form or peptidomimetic of a Reg Peptide with usage of oral interferon alfa-2a to protect beta cells from autoimmune destruction.

### Embodiment 11b

The method of any preceding embodiment, wherein the beta cells are administered to the subject through an oral, intravenous, subcutaneous, or intra-arterial route of administration with usage of oral interferon alfa-2a to protect beta cells from autoimmune destruction.

### Embodiment 11c

The method of any preceding embodiment, wherein the beta cells are delivered through the umbilical vein, portal vein, or hepatic artery.

### Embodiment 11d

The method of any preceding embodiment, wherein the beta cells are delivered directly to the pancreas or the liver with usage of oral interferon alfa-2a to protect beta cells from autoimmune destruction.

### Embodiment 11e

The method of any preceding embodiment, wherein the condition is new and existing type 1 and 2 diabetes that have characteristics of type 1 diabetes with or without autoimmune antibodies, LADA, pre-Type 1 diabetes, beta cell deficiency in which usage of oral interferon alfa-2a is used to protect beta cells from autoimmune destruction.

### Embodiment 11f

The method of any preceding embodiment, wherein the condition is associated with autoimmunity and an immune tolerance agent is administered before and/or in parallel with the administration of the beta cells and with the additional usage one or more immune agents and the use of oral interferon alfa-2a as a BRM to protect beta cells from autoimmune destruction.

### Embodiment 11g

The method of any preceding embodiment, wherein the condition is pre-Type 1 diabetes, type 1 diabetes or LADA with usage of oral interferon alfa-2a to protect beta cells from autoimmune destruction.

### Embodiment 11h

The method of any preceding embodiment, wherein the immune tolerance agent is selected from Cyclosporine, hOKT3γ1, ChAglyCD3, Rapamycin, Tacrolimus, Etanercept, Alefacept, Belatacept, Diapep277, a tuberculosis vaccine, Glutamic Acid Decarboxylase 65 (GAD65) vaccine; Bacillus Calmette-Guérin Vaccine, Mycophenolate Mofetil alone or in combination with Daclizumab; Rituximab; Campath-1H, lysofylline; antithymocyte globulin, Proleukin and the combination of Proleukin and Rapamune, Vitamin D, IBC-VSO vaccine, Ex vivo Expanded Human Autologous CD4+CD127lo/−CD25+ Polyclonal Regulatory T Cells; interferon-alfa-2a; a vaccine using CD4⁺CD25⁺ antigen-specific regulatory T cells, Interleukin-1 Receptor Antagonist (anakinra), and Alfa-2a 1-Antitrypsin.

### Embodiment 11i

The method of any preceding embodiment, wherein at least one beta regeneration or islet neogenesis agent are administered to the subject to accelerate the formation of new beta cells in the subject with additional usage of oral interferon alfa-2a to protect beta cells from autoimmune destruction.

### Embodiment 12a

The method of any preceding embodiment, wherein the subject is diabetes drug naïve or on insulin and oral interferon alfa-2a is used to protect beta cells from autoimmune destruction.

### Embodiment 12b

The composition of 22a, wherein the composition of oral interferon alfa-2a or PEGylated oral interferon alfa-2a is formulated in a pill.

### Embodiment 22b

The composition of embodiment 22a, wherein the composition wherein the composition of oral interferon alfa-2a or PEGylated oral interferon alfa-2a is formulated in a capsule.

### Embodiment 12c

The composition of embodiment 22a, wherein the composition wherein the composition of oral interferon alfa-2a or PEGylated oral interferon alfa-2a is formulated in a solution or suspension.

### Embodiment 13a

A composition comprising interferon alfa-2a or PEGylated interferon alfa-2a in a pharmaceutically acceptable carrier suitable for oral administration in a subject, wherein the interferon alfa-2a or the PEGylated interferon alfa-2a is present in the composition at an amount which is effective to protect beta cells from immune-mediated destruction in the subject but which amount does not produce one or more immunosuppressive side effects in the subject.

### Embodiment 13b

The composition of any preceding embodiment, wherein the dosage of interferon alfa-2a or PEGylated interferon alfa-2a is in the range of 1,000 to 50,000 IU.

### Embodiment 14

The composition of any preceding embodiment wherein the composition is formulated in a pill, capsule, solution, or suspension.

### Embodiment 15a

The composition of any preceding embodiment, further comprising one or more islet neogenesis or beta cell regeneration agents.

### Embodiment 15b

The composition of any preceding embodiment, wherein the one or more islet neogenesis or beta cell regeneration agents is a peptide which has an amino acid sequence set forth in any one or more of SEQ ID NOS: 1, 4, 7, 8, 11, 12, and 14.

### Embodiment 16

The composition of any preceding embodiment, wherein the peptide has been modified to increase its stability in plasma, increase its solubility, increase its protease resistance, reduce its immunogenicity, increase Tmax, and/or increase its bioavailability.

### Embodiment 17

A method of treating or preventing type I diabetes or latent autoimmune diabetes of adulthood (LADA) in a subject comprising administering to the subject: One or more islet or beta cell regeneration or replacement therapies; and a Biologic Response Modifier at an amount that is effective for protection of beta cells from immune-mediated destruction in the subject without producing one or more immunosuppressive side effects in the subject.

### Embodiment 18

The method of any preceding embodiment, wherein the Biologic Response Modifier is interferon alfa-2a or PEGylated interferon alfa-2a.

### Embodiment 19

The method of any preceding embodiment, wherein the interferon alfa-2a or the PEGylated interferon alfa-2a is administered orally to the subject.

### Embodiment 20

The method of any preceding embodiment, wherein the interferon alfa-2a or the PEGylated interferon alfa-2a is administered at a dosage in the range of 1,000 to 50,000 IU.

### Embodiment 21

The method of any preceding embodiment, wherein the one or more islet or beta cell regeneration or replacement therapies comprise one or more an islet neogenesis or beta cell regeneration agents.

### Embodiment 12

The method of any preceding embodiment, wherein the one or more islet neogenesis or beta cell regeneration agents is a peptide which has an amino acid sequence set forth in any one or more of SEQ ID NOS: 1, 4, 7, 8, 11, 12, and 14.

### Embodiment 23

The method of any preceding embodiment, wherein the peptide has been modified to increase its stability in plasma, increase its solubility, increase its protease resistance, reduce its immunogenicity, increase Tmax, and/or increase its bioavailability.

### Embodiment 24

The method of any preceding embodiment, wherein the one or more islet or beta cell regeneration or replacement therapies comprise an islet transplant, beta cell transplant, or stem cell transplant.

### Embodiment 25

The method of any preceding embodiment, wherein the one or more islet or beta cell regeneration or replacement therapies comprise one or more devices which encapsulate islets, stem cells or beta cells.

### Embodiment 26

A method of treating or preventing an autoimmune disease or condition in a subject comprising administering to the subject a Biologic Response Modifier at an amount that is effective for alleviating or preventing symptoms of the autoimmune disease or condition in the subject without producing one or more immunosuppressive side effects in the subject.

### Embodiment 27

The method of any preceding embodiment, wherein the Biologic Response Modifier is interferon alfa-2a or PEGylated interferon alfa-2a.

### Embodiment 28

The method of any preceding embodiment, wherein the interferon alfa-2a or the PEGylated interferon alfa-2a is administered orally to the subject.

### Embodiment 29

The method of any preceding embodiment, wherein the interferon alfa-2a or the PEGylated interferon alfa-2a is administered at a dosage in the range of 1,000 to 50,000 IU.

### Embodiment 30

The method of any preceding embodiment, wherein the conditions frequently seen in family members who have type 1 diabetes who have progressive diseases which may have an autoimmune basis for which there may be no treatment available and methods above may stop the progression of such autoimmune diseases or conditions that is one or more of the following: Amyotrophic Lateral Sclerosis (ALS), forms of Parkinson's disease, pulmonary fibrosis, pediatric autoimmune neurobiological disorders, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis, Juvenile Rheumatoid Arthritis and progressive neurologic conditions.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure including publications, patents, and published patent applications are each individually incorporated by reference herein in their entireties and as such are intended to provide an efficient way of supplementing the enabling disclosure of this invention as well as provide background detailing the level of ordinary skill in the art.

I claim:

1. A method of treating or preventing type 1 diabetes or latent autoimmune diabetes of adulthood (LADA) in a subject comprising administering to the subject:
One or more islet or beta cell regeneration or replacement therapies; and
A Biologic Response Modifier at an amount that is effective for protection of beta cells from immune-mediated destruction in the subject without producing one or more immunosuppressive side effects in the subject.

2. The method of claim 1, wherein the Biologic Response Modifier is interferon alfa-2a or PEGylated interferon alfa-2a.

3. The method of claim 2, wherein the interferon alfa-2a or the PEGylated interferon alfa-2a is administered orally to the subject.

4. The method of claim 2, wherein the interferon alfa-2a or the PEGylated interferon alfa-2a is administered to the subject at a dosage in the range of 1,000 to 50,000 IU.

5. The method of claim 1, wherein the one or more islet or beta cell regeneration or replacement therapies comprise one or more islet neogenesis or beta cell regeneration agents.

6. The method of claim 5, wherein the islet neogenesis or beta cell regeneration agent is a peptide which has an amino acid sequence set forth in any one or more of SEQ ID NOS: 1, 4, 7, 8, 11, 12, and 14.

7. The method of claim 1, wherein the peptide has been modified to increase its stability in plasma, increase its solubility, increase its protease resistance, reduce its immunogenicity, increase Tmax, and/or increase its bioavailability.

8. The method of claim 1, wherein the one or more islet or beta cell regeneration or replacement therapies comprise one or more of an islet transplant, beta cell transplant, or stem cell transplant.

9. The method of claim 1, wherein the one or more islet or beta cell regeneration or replacement therapies comprise one or more devices which encapsulate one or more of islets, stem cells or beta cells.

10. The method of claim 5, wherein the one or more islet neogenesis or beta cell regeneration agents comprise a small molecule or stimulatory antibody with Reg receptor binding activity.

11. The method of claim 15, wherein the small molecule or stimulatory antibody has binding activity against the peptide of SEQ ID NO:9.

12. A method of treating or preventing one or more autoimmune disease or condition in a subject comprising administering to the subject a Biologic Response Modifier at an amount that is effective for alleviating or preventing symptoms of the autoimmune disease or condition in the subject without producing one or more immunosuppressive side effects in the subject.

13. The method of claim 12, wherein conditions frequently seen in family members who have type 1 diabetes who have progressive diseases which may have an autoimmune basis for which there may be no treatment available and methods above may stop the progression of such autoimmune diseases or conditions that is one or more of Amyotrophic Lateral Sclerosis (ALS), forms of Parkinson's disease, pulmonary fibrosis, pediatric autoimmune neurobiological disorders, Myasthenia gravis, Chronic inflammatory demyelinating polyneuropathy (CIDP), Multifocal motor neuropathy (MMN), POEMS syndrome (osteosclerotic myeloma: polyneuropathy, organomegaly, endocrinopathy, monoclonal protein, skin changes), anti-myelin associated glycoprotein (MAG)-related neuropathies, Combined Sensorimotor Neuropathy in Rheumatoid Arthritis, Juvenile Rheumatoid Arthritis and progressive neurologic conditions.

14. The method of claim 12, wherein the Biologic Response Modifier is interferon alfa-2a or PEGylated interferon alfa-2a.

14. The method of claim 14, wherein the interferon alfa-2a or the PEGylated interferon alfa-2a is administered orally to the subject.

15. The method of claim 14, wherein the interferon alfa-2a or the PEGylated interferon alfa-2a is administered to the subject at a dosage in the range of 1,000 to 50,000 IU.
